(19) United States
(12) Patent Application Publication
(10) Pub. No.: US 2026/0297599 A1
(43) Pub. Date: Oct. 1, 2026

(54) LOX SITES AND METHODS FOR GENOME ENGINEERING IN PLANTS

(71) Applicant: Oregon State University, Corvallis, OR (US)

(72) Inventors: Greg Goralogia, Corvallis, OR (US); Steven H. Strauss, Corvallis, OR (US)

(73) Assignee: Oregon State University, Corvallis, OR (US)

(21) Appl. No.: 19/480,643

(22) PCT Filed: May 6, 2024
(86) PCT No.: PCT/US2024/027932

§ 371 Date: Oct. 31, 2025

Related U.S. Application Data
(60) Provisional application No. 63/500,072, filed on May 4, 2023.

(51) Int. Cl. C12N 15/82; C12N 9/12; C12N 9/22; C12N 15/11

(52) U.S. Cl. CPC C12N 15/8209; C12N 9/1241; C12N 9/226; C12N 15/111; C12N 2310/20; C12Y 207/07

(57) ABSTRACT

Embodiments of the present disclosure provide a construct and a method of using the construct to increase the efficiency of Cre-mediated transgene excision. In some embodiments, the construct comprises a cytosine-free lox recombination spacer sequence comprising a nucleotide sequence as set forth in one of SEQ ID NOs: 1-256. In other embodiments, the method is directed to increasing the efficiency of a Cre-mediated transgene excision in a plant cell, the method comprising: (a) introducing into at least one plant cell at least one plasmid comprising at least one cytosine-free lox site; and (b) contacting the at least one cytosine-free free lox site with a Cre recombinase, wherein the transgene is excised more efficiently when the at least one cytosine-free lox site is contacted with the Cre recombinase compared to transgene excision in the absence of the cytosine-free lox site.

### CROSS-REFERENCES TO RELATED APPLICATIONS

This application claims the benefit of U.S. Provisional Application No. 63/500,072, filed May 4, 2023.

### STATEMENT OF GOVERNMENT LICENSE RIGHTS

This invention was made with Government support under grant No. 2020-33522-32316, awarded by United States Department of Agriculture. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3014-P29WO_Seq_List_20240503.xml. The XML file is 318,282 bytes; was created on May 3, 2024; and is being submitted electronically via Patent Center with the filing of the specification.

### BACKGROUND

Cre recombinase is a widely used molecular tool for performing DNA manipulations both in vitro and in vivo, with applications in research and biotechnology. This includes deletion, integration, and inversion of target sequences of interest. Cre recombinase acts upon recognition sequences which are called lox sites, the most widely used of which is loxP, which is naturally present in the phage P1 genome.

Cre recombinase activity in plant somatic cells has been shown to induce gene silencing through methylation of DNA cytosine residues in inserted transgenic regions between added loxP sites. Analysis of these sites with purified DNA and Cre recombinase suggest that methylated cytosines within loxP sites themselves can hamper recombination in vitro. Reducing the plant gene silencing response had positive effects on successful DNA excision in transgenic tobacco plants.

For vegetatively propagated plants, the removal of gene editing reagents like CRISPR/Cas is difficult. In sexually propagated plant species, segregation of the stably integrated CRISPR/Cas is accomplished through segregation of the transgenic locus via mendelian inheritance while retaining desired edits. Recombinases are commonly used to delete CRISPR/Cas components or other genes useful during genetic transformation (antibiotic resistance genes and/or morphogenic regulators) after they are no longer necessary. However, the deletion of these genes in somatic tissues is currently very inefficient in plants.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides cytosine-free lox sites and methods for their use. In certain embodiments, the disclosure provides a cytosine-free lox site comprising a lox arm 1, and a lox arm 2 separated by a four to twelve nucleotide core spacer sequence. In certain specific embodiments, the core spacer sequence comprises eight nucleotides and does not include a cytosine.

In certain embodiments the cytosine-free lox site comprises a lox arm one and lox arm two comprising a nucleotide sequence SEQ ID NO:1, SEQ ID NO:2, or SEQ ID NO: 4 through SEQ ID NO:102.

In certain embodiments, the cytosine-free lox site comprises a core spacer sequence comprising randomly repeating adenine (A) and/or thymine (T) nucleotides. In certain embodiments, the core spacer sequence of the cytosine-free lox site is selected from one of AAAAAAAA (loxW10), AAAAAAAT, AAAAAATA, AAAAAATT, AAAAATAA, AAAAATAT, AAAAATTA, AAAAATTT, AAAATAAA, AAAATAAT, AAAATATA, AAAATATT (loxW9), AAAATTAA, AAAATTAT, AAAATTTA, AAAATTTT, AAATAAAA (loxW18), AAATAAAT, AAATAATA (loxW6), AAATAATT, AAATATAA, AAATATAT, AAATATTA, AAATATTT, AAATTAAA, AAATTAAT, AAATTATA, AAATTATT, AAATTTAA, AAATTTAT (loxW28), AAATTTTA, AAATTTTT, AATAAAAA, AATAAAAT (loxW21), AATAAATA, AATAAATT, AATAATAA, AATAATAT, AATAATTA, AATAATTT, AATATAAA, AATATAAT, AATATATA, AATATATT, AATATTAA, AATATTAT, AATATTTA, AATATTTT, AATTAAAA, AATTAAAT, AATTAATA, AATTAATT, AATTATAA, AATTATAT, AATTATTA, AATTATTT (loxW11), AATTTAAA, AATTTAAT, AATTTATA (loxW4), AATTTATT, AATTTTAA, AATTTTAT, AATTTTTA, AATTTTTT, ATAAAAAA (loxW15), ATAAAAAT, ATAAAATA (loxW22), ATAAAATT, ATAAATAA, ATAAATAT, ATAAATTA, ATAAATTT, ATAATAAA, ATAATAAT, ATAATATA, ATAATATT (loxW26), ATAATTAA (loxW17), ATAATTAT, ATAATTTA, ATAATTTT, ATATAAAA, ATATAAAT, ATATAATA, ATATAATT, ATATATAA, ATTTAATA, ATATATAT, ATATATTA, ATATATTT, ATATTAAA (loxW8), ATATTAAT, ATATTATA, ATATTATT, ATATTTAA, ATATTTAT, ATATTTTA, ATATTTTT, ATTAAAAA, ATTAAAAT, ATTAAATA, ATTAAATT, ATTAATAA, ATTAATAT, ATTAATTA, ATTAATTT, ATTATAAA, ATTATAAT, ATTATATA, ATTATATT, ATTATTAA, ATTATTAT, ATTATTTA, ATTATTTT, ATTTAAAA, ATTTAAAT (loxW25), TAAATTTT, ATTTAATT, ATTTATAA, ATTTATAT (loxW20), ATTTATTA, ATTTATTT, ATTTTAAA, ATTTTAAT, ATTTTATA, ATTTTATT, ATTTTTAA, ATTTTTAT, ATTTTTTA, ATTTTTTT, TAAAAAAA, TAAAAAAT, TAAAAATA, TAAAAATT, TAAAATAA, TAAAATAT, TAAAATTA (loxW12), TAAAATTT, TAAATAAA, TAAATAAT, TAAATATA, TAAATATT, TAAATTAA, TAAATTAT, TAAATTTA, TATATTAA, TATATTAT, TAATAAAA, TAATAAAT, TAATAATA, TAATAATT, TAATATAA, TAATATAT, TAATATTA, TAATATTT, TAATTAAA, TAATTAAT, TAATTATA, TAATTATT, TAATTTAA, TAATTTAT, TAATTTTA, TAATTTTT, TATAAAAA, TATAAAAT, TATAAATA, TATAAATT, TATAATAA, TATAATAT, TATAATTA, TATAATTT, TATATAAA, TATATAAT, TATATATA, TATATATT (loxW7), TTAATAAT, TTAATATA, TATATTTA, TATATTTT, TATTAAAA, TATTAAAT, TATTAATA, TATTAATT, TATTATAA, TATTATAT, TATTATTA, TATTATTT (loxW24), TATTTAAA, TATTTAAT, TATTTATA, TATTTATT, TATTTTAA, TATTTTAT (lox W5), TATTTTTA, TATTTTTT, TTAAAAAA, TTAAAAAT (lox W23), TTAAAATA, TTAAAATT, TTAAATAA, TTAAATAT, TTAAATTA, TTAAATTT, TTAATAAA, TTTAATTA, TTTAATTT, TTTATAAA, TTAATATT, TTAATTAA, TTAATTAT, TTAATTTA (loxW19), TTAATTTT (loxW2), TTATAAAA, TTATAAAT, TTATAATA, TTATAATT, TTATATAA, TTATATAT, TTATATTA, TTATATTT, TTATTAAA, TTATTAAT, TTATTATA, TTATTATT, TTATTTAA (loxW1), TTATTTAT, TTATTTTA, TTATTTTT, TTTAAAAA, TTTAAAAT, TTTAAATA, TTTAAATT, TTTAATAA, TTTAATAT, TTTTAATT, TTTTATAA, TTTTATAT, TTTATAAT, TTTATATA, TTTATATT, TTTATTAA, TTTATTAT, TTTATTTA, TTTATTTT, TTTTAAAA, TTTTAAAT, TTTTAATA, TTTTATTA, TTTTATTT (loxW3), TTTTTAAA, TTTTTAAT, TTTTTATA, TTTTTATT, TTTTTTAA, TTTTTTAT, TTTTTTTA, or TTTTTTTT.

In certain embodiments the cytosine-free lox site selected from one of as set forth in one of SEQ ID NO: 103-SEQ ID NO:357.

The present disclosure also provides a method to increase the efficiency of Cre-mediated transgene excision in a plant cell, the method comprising: (a) introducing into at least one plant cell at least one plasmid comprising at least one cytosine-free lox site according to any of the above embodiments; and (b) contacting the at least one cytosine-free free lox site with a Cre recombinase; wherein the transgene is excised more efficiently when the at least one cytosine-free lox site is contacted with the Cre recombinase compared to transgene excision in the absence of the cytosine-free lox site.

In certain embodiments the method comprises the use of a plasmid comprises a transgene flanked by direct repeats of the cytosine-free lox site. In certain embodiments the transgene comprises a gene encoding Cas 9 and at least one guide RNA. In certain embodiments, the plasmid further comprises a Cre coding region. In certain embodiments the cytosine-free lox site is selected from a sequence as set forth in any one of SEQ ID NO: 103-SEQ ID NO:357.

In certain embodiments, the plasmid comprises a constitutive promoter, an inducible promoter, or a tissue specific promoter controlling Cre expression. In certain embodiments, the plasmid comprises a reporter gene. In certain more specific embodiments the reporter is a fluorescent or DNA methylation sensitive reporter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 depicts how steric hinderance of methylated cytosine residues in the wild-type loxP sequence can impair Cre recombinase activity in vitro (Liu et al., DNA methylation occurring in Cre-expressing cells inhibits loxP recombination and silences loxP-sandwiched genes. New Phytol. 231 (1): 210-224, 2021). The present disclosure demonstrates the discovery and use of recombination sites whose core spacer sequences lack cytosines that improve excision efficiency in plants.

FIG. 2 depicts the strategy used for finding and validating cytosine-free recombination sites. LoxP arm 1 (SEQ ID NO:1) and LoxP arm 2 (SEQ ID NO: 2) are demonstrated. Location of successful excision footprints shown with left pointing arrow or Cre.

FIG. 3 shows that cytosine-free lox sites were more efficient in excision than wild-type loxP sequences. Leaves from tobacco were imaged 4 days after agroinfiltration. Strong reductions of tdTomato (an exceptionally bright red fluorescent protein) expression can be seen in Cre-containing constructs (bottom panels) relative to a no-Cre control (top panels), which could be due to excision of the tdTomato fragment or silencing without excision. However, use of the disclosed fluorescent-switch-reporter constructs (that enable green fluorescent protein (GFP) expression after excision) verified that excision was occurring, and that the cytosine-free LoxP core site of LoxW7 (TATATATT)-containing leaves were more effective than native LoxP in supporting excision (Bottom panel).

FIGS. 4A and 4B demonstrate that cytosine-free lox sites were more efficient at inducing successful excision in stable poplar transgenics. FIG. 4A shows LoxP and LoxW7-containing transgenes with constitutive Cre and a methylation-sensitive excision reporter exhibit reduced shoot regeneration rates vs. no Cre containing controls in poplar, but LoxW7 transgenes allow (approximately 10%) excised shoot recovery. FIG. 4B shows LoxP and LoxW7-containing transgenes with constitutive Cre and methylation-resistant excision reporter (26% GC content proximal to Lox sites) substantially reduce LoxW7 negative-regeneration effects but not LoxP. Using this reporter, LoxW7 sites resulted in greater recovery of excised shoots versus LoxP.

FIG. 5 shows a plant transformation vector example utilizing cytosine-free lox sites to enable CRISPR and/or morphogenic gene excision after transformation. Cytosine-free lox sites (as an example, the loxW7 site is designated) were added in direct repeats in the flanking arms of the T-DNA, 100-300 base pairs inboard of the T-DNA left and right borders. After excision, only a small piece of residual inserted DNA remains in the genome comprising a lox site and the left and right border regions. (bottom construct diagram).

FIG. 6 shows LoxW7-containing transgenes with heat-shock inducible Cre and a methylation-resistant excision reporter do not pre-excise early in transgenesis, allowing for dynamic control of excision via heat shock during transformation.

FIGS. 7A through 7C show LoxW7-containing transgenes with heat-shock inducible Cre, gene editing components and DEV genes result in efficient transgene excision in poplar. FIG. 7A provides a map of the vector construct. FIG. 7B depicts regenerated shoots that demonstrate excision of the transgene and examples of developing plants with the RUBY transgene and with the RUBY transgene excised.

FIG. 7C shows the percent of excision events with and without heat shock and the transgenic efficiency of excision at the DNA level. Forty eight percent (48%) of events yielded visually excised propagules after two weeks of heat shock treatment and one month on SIM (shoot induction media), and 50% of those propagules were verified to be transgene-free by PCR (FIG. 7C).

### DETAILED DESCRIPTION

The process of transgene excision would be greatly aided by improved efficiency of recombinases for their target sites in plants. The present disclosure provides recombination sites for the most widely used recombinase which facilitate increased plant activity by making them resistant to cytosine methylation which occurs in unique contexts in plants.

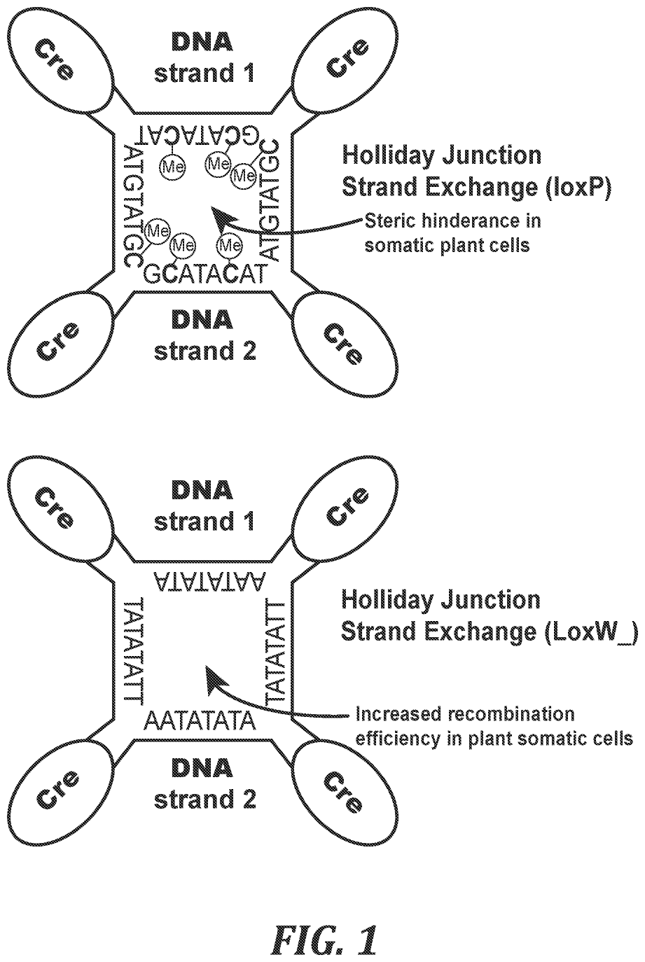

In embodiments, the present disclosure describes cytosine-free lox sequences, and a method for generating them, which were designed to increase Cre-mediated excision rates on their respective lox sites specifically in plant cells. Because plants have different methods and contexts of DNA methylation compared with animals, cytosine methylation prevents efficient recombination of target sites (FIG. 1). Discovery and implementation of lox sites which are resistant to cytosine methylation improves their ability to be acted on successfully by Cre recombinase when Cre is expressed. Although many alternative lox sites have been discovered through previous screening strategies, none are cytosine-free in the spacer sequence nor are any optimized for use as excision tools in plants.

### Method of Identifying Methylation Resistant Lox Sites Lacking Cytosine

The present disclosure provides a unique screening method to identify lox sites located in the recombination core spacer sequence and that lack cytosines and are therefore less or non-susceptible to methylation in plants.

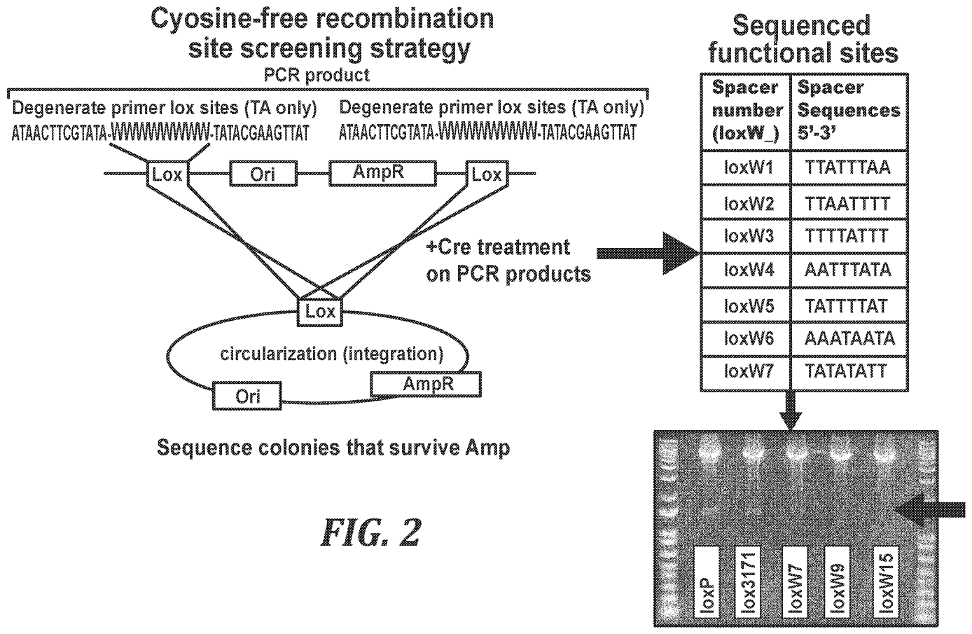

In embodiments, this method leverages flanking degenerate primer sequences (both 3′ and 5′) whose overhangs include lox sites with spacers which only contain the DNA bases A or T for use in PCR (polymerase chain reaction). Amplification of a linear product containing a bacterial origin sequence and an antibiotic resistance gene was undertaken (FIG. 2). These PCR products were treated in vitro with purified Cre recombinase, and those with compatible lox sites with cytosine-free spacer sequences circularized into plasmids capable of replication and selection in E. coli. Colonies produced through this method were sequenced. The resulting lox sites with cytosine-free core spacer regions were screened for activity through cloning two direct repeats in plasmids and treating with purified Cre recombinase for excision products.

### Increased Recombination Efficiency in Plants

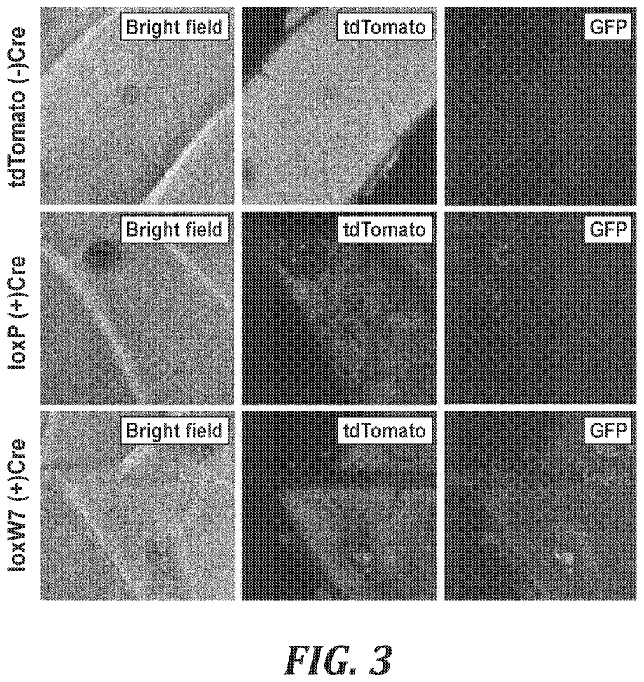

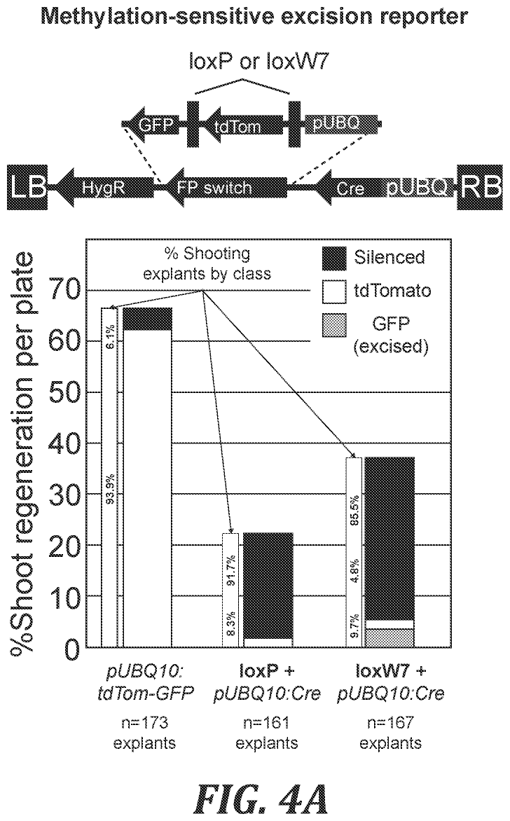

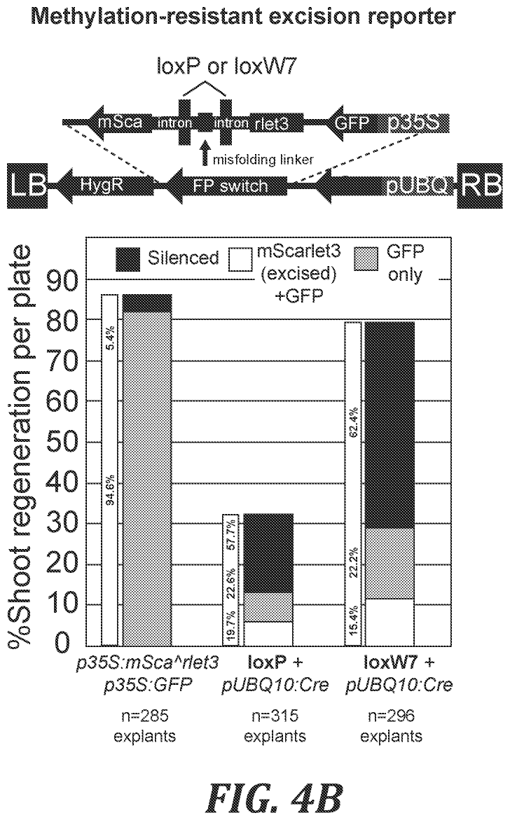

To implement these lox sequences and test for increased recombination efficiency in plants, well-performing sites were cloned in direct repeats, the first placed in the 5′ UTR of a fluorescent reporter gene, and the second placed after the terminator of the same fluorescent gene. A second fluorescent reporter was placed downstream of this site without a promoter such that excision via Cre of the first product turns on expression of the second reporter (FIG. 3). Testing of these vectors with a constitutively expressed Cre in tobacco agroinfiltration assays (Schöb et al. Mol. Genet. Genomics 256:581-585, 1997; Norkunas et al. Plant Methods 14:71, 2018; both incorporated herein by reference) showed significantly increased transgene excision with cytosine-free spacer lox sites relative to the same vector with traditional loxP sites. The same result was found in stable transgenic calli and shoots in the hybrid poplar P. tremula x alba using constitutive Cre recombinase (FIGS. 4A and 4B).

### Uses of the Cytosine Free Lox Sites

Possible uses for the disclosed methods and lox sites include excision of morphogenic genes used in plant transformation and gene editing, excision of gene editing genes, site-specific integration of transgenes, development of biosensors or inducible switches using Cre recombinase in plants, and fate-mapping tools for use in plants. Use of these cytosine free lox sites is conventional for one skilled in the art with existing strategies using loxP, such as providing direct repeats of loxP sites flanking desired segments to be deleted after transformation, or during the induction of a genetic switch. Conversely, Cre recombinase will act upon a single site at a previously inserted locus should gene integration be desired. Cre recombinase expression control is conventional with cytosine-free sites, however due to the increased rates of Cre activity (e.g., excision), additional control of baseline Cre recombinase expression may be desirable based on the somatic or germline cell type of interest where a recombination event is to be triggered.

### Vectors Containing Lox Sequences

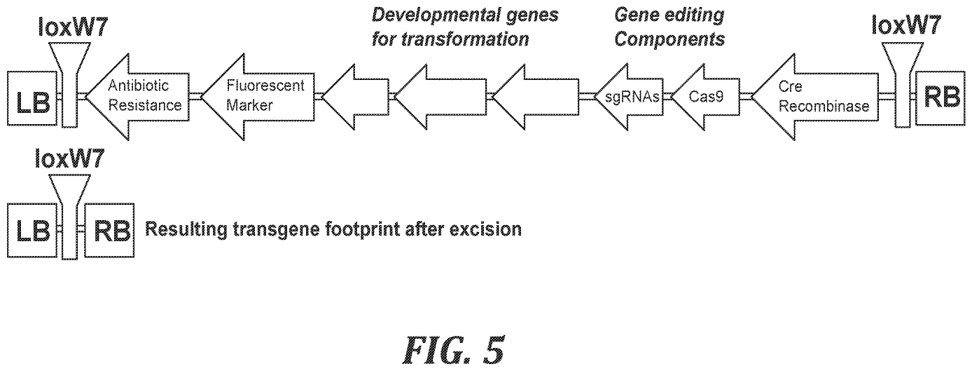

For the deletion of gene editing and morphogenic genes after the initial transformation step using Cre recombinase and cytosine-free lox sites, vectors which are flanked by direct repeats of the lox sites, between the T-DNA left and right borders (FIG. 5). These are placed 100-300 base pairs inboard of these locations to allow successful excision in cases where T-DNA insertion fidelity is imperfect and is truncated relative to the T-DNA left or right border. The remaining “footprint” of DNA remaining in the genome is then generally less than 500 base pairs and contains only non-coding DNA elements including an intact cytosine-fee lox site unless a transgene is desired to be inserted at the same time as gene editing is to commence. Cytosine-free lox sites can be added to existing T-DNA vectors in a conventional PCR-based approach via long primers, Gibson assembly, oligo annealing, or other common cloning techniques to those skilled in the art. (Han et al. Plant Cell Rep. 19:315-320, 2000; Birch Nuc. Acids Res. 26(11):2729-2734, 1998; both incorporated herein in their entirety).

In accordance with the foregoing, in one aspect, the disclosure provides for a cytosine-free lox recombination core spacer sequence comprising a nucleotide sequence as set forth in one of the nucleotide sequences in Table 2.

In another aspect, the disclosure provides for a method to increase the efficiency of Cre recombinase-mediated transgene excision in a plant cell, the method comprising: (a) introducing into at least one plant cell at least one plasmid comprising at least one cytosine-free lox site; and (b) contacting the at least one cytosine-free free lox site with a Cre recombinase, wherein the transgene is excised more efficiently when the at least one cytosine-free lox site is contacted with the Cre recombinase compared to transgene excision in the absence of the cytosine-free lox site.

In some embodiments, the plasmid comprises a transgene flanked by direct repeats of the cytosine-free lox site. See for example, as specific embodiments FIG. 4A and FIG. 4B which provide examples of molecular switches.

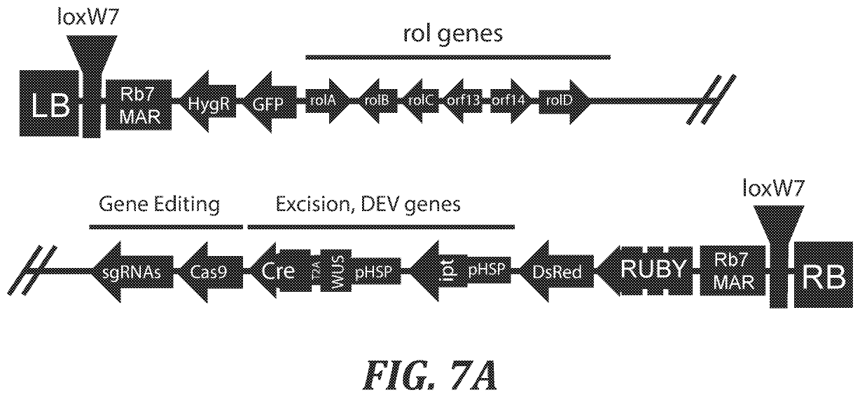

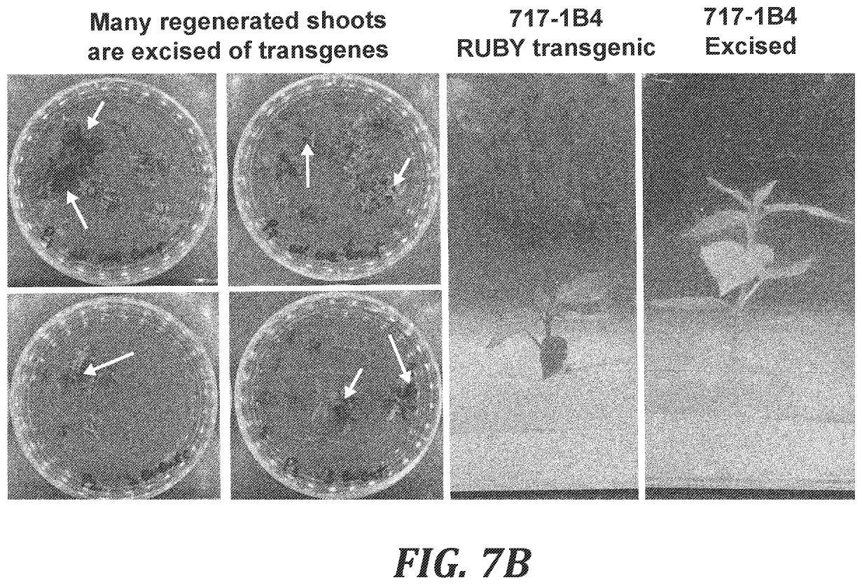

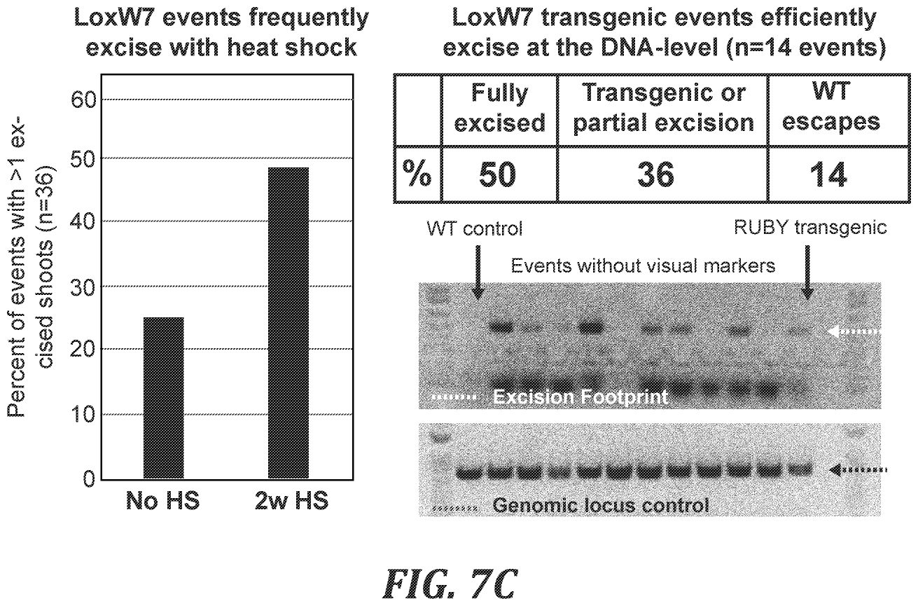

In other embodiments, the plasmid further comprises a Cre recombinase coding region. See for example, as specific embodiments FIG. 5 and FIG. 7A.

In some embodiments, the cytosine-free lox site comprises a Cre recognition sequence, and its palindrome selected from a sequence as set forth in any one of SEQ ID NOs: 1, 2, and 4 through 102 separated by a 4 to 12 base pair cytosine-free core spacer sequence.

In other embodiments, the cytosine-free lox site comprises an eight base pair core spacer sequence comprising randomly repeating adenine (A) and/or thymine (T) nucleotides. In some embodiments, the core sequence comprises between 4 to 12 randomly repeated A and/or T nucleotides.

In certain embodiments, the cytosine-free lox site comprises a loxP recognition sequence and its palindrome separated by an eight base pair cytosine-free nucleotide core spacer sequence wherein the loxP cytosine-free site is selected from one of the nucleotide sequences SEQ ID NO:103-357.

### Additional Definitions

As used herein, “transformation” means processes by which cells/tissues/plants acquire properties encoded on a nucleic acid molecule that has been transferred to the cell/tissue/plant. “Transferring” refers to methods to transfer DNA into cells including, but not limited to, microinjection, permeabilizing the cell membrane with various physical (e.g., electroporation) or chemical (e.g., polyethylene glycol, PEG) treatments, high-velocity microprojectile bombardment also termed biolistics, or infection with Agrobacterium tumefaciens or A. rhizogenes. In some embodiments, the exogenously supplied DNA is either T-DNA, PCR-derived, or plasmid-derived.

As used herein, “transformant” means a plant which has acquired properties encoded on a nucleic acid molecule that has been transferred to cells during the process known as transformation. As used herein, “re-transformation” means transformation of cells/tissues/plants which are in themselves transformants.

As used herein, Cre recombinase-mediated transgene excision refers to but is not limited to: (i) deletion of a pre-selected DNA segment flanked by lox sites; (ii) inversion of the nucleotide sequence of a pre-selected DNA segment flanked by lox sites; and (iii) reciprocal exchange of DNA segments proximate to lox sites on different DNA molecules. Subsequent to excision a cytosine-free lox site remains after excision of the selected transgene.

The expression “nucleotide sequence,” as used herein refers to a polymer of DNA or RNA, which can be single- or double-stranded, optionally containing synthetic, non-natural, or altered nucleotides capable of incorporation into DNA or RNA polymers. A “DNA segment” refers to a linear fragment of single- or double-stranded DNA, which can be derived from any source. The expression “DNA in plant cells” includes all DNA present in plant cells.

A “coding region” refers to a DNA segment which encodes a regulatory molecule or any polypeptide. The expression “regulatory molecule” refers to a polymer of ribonucleic acid (RNA), such as antisense RNA or a ribozyme, or a polypeptide which is capable of enhancing or inhibiting expression of a gene product. The term “expression” refers to the synthesis of gene product from a gene coding for the sequence of the gene product. The gene product can be a RNA or a protein.

As used herein, the term “promoter region” refers to a sequence of DNA, usually upstream (5′) of the coding sequence, which controls the expression of the coding region by providing the recognition for RNA polymerase and/or other factors required for transcription to start at the correct site. A “promoter fragment” constitutes a DNA sequence consisting of the promoter region. A promoter region can include one or more regions which control the effectiveness of transcription initiation in response to physiological conditions (e.g., an inducible promoter), and a transcription initiation sequence. A “tissue specific promoter” as referred to herein is one that directs gene expression primarily in specific tissues such as roots, leaves, stems, pistils, anthers, flower petals, seed coat, seed nucellus or epidermal layers. Transcription stimulators, enhancers or activators may be integrated into tissue specific promoters to create a promoter with a high level of activity that retains tissue specificity.

“Introducing” as used herein is intended to mean presenting to the organism, such as a plant, or the cell a polynucleotide or polypeptide in such a manner that the polynucleotide or polypeptide comprising a sequence of nucleotides or amino acids gains access to the interior of a cell of the organism or to the cell itself. The methods and compositions do not depend on a particular method for introducing a polynucleotide or polypeptide into an organism or cell, only that the polynucleotide or polypeptide gains access to the interior of at least one cell of the organism. Methods for introducing polynucleotides or polypeptides into plants are well known in the art and include, but not limited to, stable transformation methods, transient transformation methods, virus-mediated methods, and sexual breeding.

“Stable transformation” as used herein is intended to mean that the nucleotide construct introduced into a plant integrates into a genome of the plant and is capable of being inherited by the progeny thereof. “Transient transformation” as used herein is intended to mean that a polynucleotide is introduced into the plant and does not integrate into a genome of the plant, or a polypeptide is introduced into a plant.

As used herein a “transgene” is a gene which is introduced into the genome of a plant cell either artificially or naturally. A “transgene of interest” is a gene that is selected for a particular purpose to be introduced into the plant cell. The method of introduction can include any number of genetic engineering techniques well known in the art.

The wild-type recognition site on the bacteriophage P1 recognized by Cre is a 34 base pair (bp) double stranded DNA sequence known as LoxP. The LoxP site is palindromic with the exception of its eight innermost base pairs, which impart directionality to the site. As used herein, a complete single cytosine free lox site comprises the following configuration: lox arm 1—core spacer sequence—lox arm 2; wherein lox arm 1 is the 5′ Cre recognition sequence and lox arm 2 is the Cre 3′ recognition sequence. The wild-type core spacer sequence is the nucleotide sequence GCATACAT. In some particular embodiments, a complete single cytosine free lox site comprises the configuration LoxP arm 1—core spacer sequence—LoxP arm 2. In some embodiments, a complete cytosine free lox site flanks each end of the transgene of interest to be deleted. In some embodiments, the complete cytosine free lox site flanking each end of a transgene of interest to be deleted comprises identical sequences (i.e., each sequence is the same). In some embodiments, the complete cytosine free lox site flanking each end of the transgene of interest to be deleted comprises different sequences (i.e, each sequence is different). One skilled in the art would understand that for some applications, it can be advantageous if sites had very low promiscuity with divergent sequences, for example, if there were more than two complete single cytosine free lox sites in the construct and they were to operate in pairs.

The Cre recombinase has been shown to be active on various nucleotide sequences that vary considerably from the bacteriophage recognition sequence LoxP (LoxP arm 1—ATAACTTCGTATA (SEQ ID NO: 1); LoxP arm 2—TATACGAAGTTAT (SEQ ID NO: 2)). The nucleotide sequences of the alternative recognition sites are provided below and derivation of these sequences to form full length cytosine-free lox sites is considered part of the presently disclosed compositions and methods. The alternative lox recognition sequences (lox arm 1 and lox arm 2 are provided in Table 1 and are as follows:

TABLE 1
Alternative Cre recognition sequences.

| Lox arm 1 | Lox arm 2 |
|---|---|
| ACTATGTCGCACT (SEQ ID NO: 3) | AGTGCGACATAGT (SEQ ID NO: 4) |
| GAAAGGGGCTATA (SEQ ID NO: 5) | TATAGCCCCTTTC (SEQ ID NO: 6) |
| CTGCAATCTCATT (SEQ ID NO: 7) | AATGAGATTGCAG (SEQ ID NO: 8) |
| GCACCAGCCTATC (SEQ ID NO: 9) | GATAGGCTGGTGC (SEQ ID NO: 10) |
| CAAAATCCGGAAA (SEQ ID NO: 11) | TTTCCGGATTTTG (SEQ ID NO: 12) |
| CTAGCCACAATAA (SEQ ID NO: 13) | TTATTGTGGCTAG (SEQ ID NO: 14) |
| ACAGAAACGCGTT (SEQ ID NO: 15) | AACGCGTTTCTGT (SEQ ID NO: 16) |
| AACAAGTCATATA (SEQ ID NO: 17) | TATATGACTTGTT (SEQ ID NO: 18) |
| CAAGTTTCAATAA (SEQ ID NO: 19) | TTATTGAAACTTG (SEQ ID NO: 20) |
| AACCGATCCCCTC (SEQ ID NO: 21) | GAGGGGATCGGTT (SEQ ID NO: 22) |
| AAAACCCCCATAA (SEQ ID NO: 23) | TTATGGGGGTTTT (SEQ ID NO: 24) |
| CACTGGCCGTATC (SEQ ID NO: 25) | GATACGGCCAGTG (SEQ ID NO: 26) |
| TTACTTGAACCAC (SEQ ID NO: 27) | GTGGTTCAAGTAA (SEQ ID NO: 28) |
| CACCATGTGTATT (SEQ ID NO: 29) | AATACACATGGTG (SEQ ID NO: 30) |
| TGGATCCGAAATA (SEQ ID NO: 31) | TATTTCGGATCCA (SEQ ID NO: 32) |
| ATCACGATGACTT (SEQ ID NO: 33) | AAGTCATCGTGAT (SEQ ID NO: 34) |
| AATATATCAACTG (SEQ ID NO: 35) | CAGTTGATATATT (SEQ ID NO: 36) |
| AAACACGCCAACA (SEQ ID NO: 37) | TGTTGGCGTGTTT (SEQ ID NO: 38) |
| TTGCCACGGCTAA (SEQ ID NO: 39) | TTAGCCGTGGCAA (SEQ ID NO: 40) |
| CTCACTACCAAAA (SEQ ID NO: 41) | TTTTGGTAGTGAG (SEQ ID NO: 42) |
| ATAACACACCAAT (SEQ ID NO: 43) | ATTGGTGTGTTAT (SEQ ID NO: 44) |
| AACACCACCGATA (SEQ ID NO: 45) | TATCGGTGGTGTT (SEQ ID NO: 46) |
| AAGCAAGCCATTA (SEQ ID NO: 47) | TAATGGCTTGCTT (SEQ ID NO: 48) |
| CAACACAAAAATA (SEQ ID NO: 49) | TATTTTTGTGTTG (SEQ ID NO: 50) |
| TCTCCACACATAG (SEQ ID NO: 51) | CTATGTGTGGAGA (SEQ ID NO: 52) |
| TAAAAACGACATA (SEQ ID NO: 53) | TATGTCGTTTTTA (SEQ ID NO: 54) |
| ATAAACCCACTCA (SEQ ID NO: 55) | TGAGTGGGTTTAT (SEQ ID NO: 56) |
| ATCGCAGTCTACG (SEQ ID NO: 57) | CGTAGACTGCGAT (SEQ ID NO: 58) |
| CTAAGGACGCATA (SEQ ID NO: 59) | TATGCGTCCTTAG (SEQ ID NO: 60) |
| ATGGGCCCAGATA (SEQ ID NO: 61) | TATCTGGGCCCAT (SEQ ID NO: 62) |
| ACAACCTCCCTTA (SEQ ID NO: 63) | TAAGGGAGGTTGT (SEQ ID NO: 64) |
| CAAACACCCGGCA (SEQ ID NO: 65) | TGCCGGGTGTTTG (SEQ ID NO: 66) |
| CCACCTACAGAGA (SEQ ID NO: 67) | TCTCTGTAGGTGG (SEQ ID NO: 68) |
| TGAACGCAAGAAA (SEQ ID NO: 69) | TTTCTTGCGTTCA (SEQ ID NO: 70) |
| ATACTTACACAAT (SEQ ID NO: 71) | ATTGTGTAAGTAT (SEQ ID NO: 72) |
| AAACAATCCCATG (SEQ ID NO: 73) | CATGGGATTGTTT (SEQ ID NO: 74) |
| ACAGCCTCGGAGT (SEQ ID NO: 75) | ACTCCGAGGCTGT (SEQ ID NO: 76) |
| AAAGCTCCTACTA (SEQ ID NO: 77) | TAGTAGGAGCTTT (SEQ ID NO: 78) |
| AACCACAGTATAA (SEQ ID NO: 79) | TTATACTGTGGTT (SEQ ID NO: 80) |
| AAAGCGCCCGGTA (SEQ ID NO: 81) | TACCGGGCGCTTT (SEQ ID NO: 82) |
| TTAGATAGCGAGA (SEQ ID NO: 83) | TCTCGCTATCTAA (SEQ ID NO: 84) |
| ATAACTTCATATA (SEQ ID NO: 85) | TATATGAAGTTAT (SEQ ID NO: 86) |
| ATAACTTCGTACA (SEQ ID NO: 87) | TGTACGAAGTTAT (SEQ ID NO: 88) |
| ATAACTTCATACA (SEQ ID NO: 89) | TGTATGAAGTTAT (SEQ ID NO: 90) |
| ATAACTTTGTATA (SEQ ID NO: 91) | TATACAAAGTTAT (SEQ ID NO: 92) |
| ATAACTTCGTGCA (SEQ ID NO: 93) | TGCACGAAGTTAT (SEQ ID NO: 94) |
| ATAACTCTGTATA (SEQ ID NO: 95) | TATACAGAGTTAT (SEQ ID NO: 96) |
| ATAACTCTATATA (SEQ ID NO: 97) | TATATAGAGTTAT (SEQ ID NO: 98) |
| ATAACTCTGTGTA (SEQ ID NO: 99) | TACACAGAGTTAT (SEQ ID NO: 100) |
| TACCGTTCGTATA (SEQ ID NO: 101) | TATACGAACGGTA (SEQ ID NO: 102) |

### Nucleotide Sequences for Cytosine-Free Recombination

The following nucleotide sequences can be useful in practicing the various embodiments and broader concepts of the present disclosure and envisioned herein.

As above, the structure of the cytosine-free Cre recombinase recognition sites have the basic structure of lox arm 1-core spacer sequence-lox arm 2. Lox arm 2 is the reverse nucleotide sequence lox arm 1. In specific embodiments of the cytosine-free Cre recombinase recognition the nucleotide sequences comprise LoxP arm 1—core spacer sequence—LoxP arm 2. The nucleotide sequences of the cytosine-free core spacer sequences are provided below in Table 2.

TABLE 2
Cytosine-free recombination core sequences

| | |
|---|---|
| AAAAAAAA (loxW10) | AAATTTAA |
| AAAAAAAT | AAATTTAT (loxW28) |
| AAAAAATA | AAATTTTA |
| AAAAAATT | AAATTTTT |
| AAAAATAA | AATAAAAA |
| AAAAATAT | AATAAAAT (loxW21) |
| AAAAATTA | AATAAATA |
| AAAAATTT | AATAAATT |
| AAAATAAA | AATAATAA |
| AAAATAAT | AATAATAT |
| AAAATATA | AATAATTA |
| AAAATATT (loxW9) | AATAATTT |
| AAAATTAA | AATATAAA |
| AAAATTAT | AATATAAT |
| AAAATTTA | AATATATA |
| AAAATTTT | AATATATT |
| AAATAAAA (loxW18) | AATATTAA |
| AAATAAAT | AATATTAT |
| AAATAATA (loxW6) | AATATTTA |
| AAATAATT | AATATTTT |
| AAATATAA | AATTAAAA |
| AAATATAT | AATTAAAT |
| AAATATTA | AATTAATA |
| AAATATTT | AATTAATT |
| AAATTAAA | AATTATAA |
| AAATTAAT | AATTATAT |
| AAATTATA | AATTATTA |
| AAATTATT | AATTATTT (loxW11) |
| AATTTAAA | ATATATAT |
| AATTTAAT | ATATATTA |
| AATTTATA (loxW4) | ATATATTT |
| AATTTATT | ATATTAAA (loxW8) |
| AATTTTAA | ATATTAAT |
| AATTTTAT | ATATTATA |
| AATTTTTA | ATATTATT |
| AATTTTTT | ATATTTAA |
| ATAAAAAA (loxW15) | ATATTTAT |
| ATAAAAAT | ATATTTTA |
| ATAAAATA (loxW22) | ATATTTTT |
| ATAAAATT | ATTAAAAA |
| ATAAATAA | ATTAAAAT |
| ATAAATAT | ATTAAATA |
| ATAAATTA | ATTAAATT |
| ATAAATTT | ATTAATAA |
| ATAATAAA | ATTAATAT |
| ATAATAAT | ATTAATTA |
| ATAATATA | ATTAATTT |
| ATAATATT (loxW26) | ATTATAAA |
| ATAATTAA (loxW17) | ATTATAAT |
| ATAATTAT | ATTATATA |
| ATAATTTA | ATTATATT |
| ATAATTTT | ATTATTAA |
| ATATAAAA | ATTATTAT |
| ATATAAAT | ATTATTTA |
| ATATAATA | ATTATTTT |
| ATATAATT | ATTTAAAA |
| ATATATAA | ATTTAAAT (loxW25) |
| ATTTAATA | TAAATTTT |
| ATTTAATT | TAATAAAA |
| ATTTATAA | TAATAAAT |
| ATTTATAT (loxW20) | TAATAATA |
| ATTTATTA | TAATAATT |
| ATTTATTT | TAATATAA |
| ATTTTAAA | TAATATAT |
| ATTTTAAT | TAATATTA |
| ATTTTATA | TAATATTT |
| ATTTTATT | TAATTAAA |
| ATTTTTAA | TAATTAAT |
| ATTTTTAT | TAATTATA |
| ATTTTTTA | TAATTATT |
| ATTTTTTT | TAATTTAA |
| TAAAAAAA | TAATTTAT |
| TAAAAAAT | TAATTTTA |
| TAAAAATA | TAATTTTT |
| TAAAAATT | TATAAAAA |
| TAAAATAA | TATAAAAT |
| TAAAATAT | TATAAATA |
| TAAAATTA (loxW12) | TATAAATT |
| TAAAATTT | TATAATAA |
| TAAATAAA | TATAATAT |
| TAAATAAT | TATAATTA |
| TAAATATA | TATAATTT |
| TAAATATT | TATATAAA |
| TAAATTAA | TATATAAT |
| TAAATTAT | TATATATA |
| TAAATTTA | TATATATT (loxW7) |
| TATATTAA | TTAATAAT |
| TATATTAT | TTAATATA |
| TATATTTA | TTAATATT |
| TATATTTT | TTAATTAA |
| TATTAAAA | TTAATTAT |
| TATTAAAT | TTAATTTA (loxW19) |
| TATTAATA | TTAATTTT (loxW2) |
| TATTAATT | TTATAAAA |
| TATTATAA | TTATAAAT |
| TATTATAT | TTATAATA |
| TATTATTA | TTATAATT |
| TATTATTT (loxW24) | TTATATAA |
| TATTTAAA | TTATATAT |
| TATTTAAT | TTATATTA |
| TATTTATA | TTATATTT |
| TATTTATT | TTATTAAA |
| TATTTTAA | TTATTAAT |
| TATTTTAT (loxW5) | TTATTATA |
| TATTTTTA | TTATTATT |
| TATTTTTT | TTATTTAA (loxW1) |
| TTAAAAAA | TTATTTAT |
| TTAAAAAT (loxW23) | TTATTTTA |
| TTAAAATA | TTATTTTT |
| TTAAAATT | TTTAAAAA |
| TTAAATAA | TTTAAAAT |
| TTAAATAT | TTTAAATA |
| TTAAATTA | TTTAAATT |
| TTAAATTT | TTTAATAA |
| TTAATAAA | TTTAATAT |
| TTTAATTA | TTTTAATT |
| TTTAATTT | TTTTATAA |
| TTTATAAA | TTTTATAT |
| TTTATAAT | TTTTATTA |
| TTTATATA | TTTTATTT (loxW3) |
| TTTATATT | TTTTTAAA |
| TTTATTAA | TTTTTAAT |
| TTTATTAT | TTTTTATA |
| TTTATTTA | TTTTTATT |
| TTTATTTT | TTTTTTAA |
| TTTTAAAA | TTTTTTAT |
| TTTTAAAT | TTTTTTTA |
| TTTTAATA | TTTTTTTT |

Representative combined LoxP arm 1—cysteine-free core sequence—LoxP arm 2 nucleotide sequences are as follows: ATAACTTCGTATAAAAAAAAATAT ACGAAGTTAT (SEQ ID NO: 103) designated loxW10; comprising LoxP arm 1 (SEQ ID NO: 1) the core spacer sequence AAAAAAAA and LoxP arm 2 (SEQ ID NO:2); and ATAACTTCGTATA TATATATTT ATACGAAGTTAT (SEQ ID NO:273) designated loxW7, comprising LoxP arm 1 (SEQ ID NO:1) the core sequence TATATATT, and LoxP arm 2 (SEQ ID NO:2).

TABLE 3
Combined lox recognition sequences and cytosine
free core spacer sequences

| Combined loxP-cytosine free | |
|---|---|
| core spacer sequence (full length) | SEQ ID NO: |
| ATAACTTCGTATAAAAAAAAATATACGAAGTTAT | 103 |
| ATAACTTCGTATAAAAAAAATTATACGAAGTTAT | 104 |
| ATAACTTCGTATAAAAAAATATATACGAAGTTAT | 105 |
| ATAACTTCGTATAAAAAAATTTATACGAAGTTAT | 106 |
| ATAACTTCGTATAAAAAATAATATACGAAGTTAT | 107 |
| ATAACTTCGTATAAAAAATATTATACGAAGTTAT | 108 |
| ATAACTTCGTATAAAAAATTATATACGAAGTTAT | 109 |
| ATAACTTCGTATAAAAAATTTTATACGAAGTTAT | 110 |
| ATAACTTCGTATAAAAATAAATATACGAAGTTAT | 111 |
| ATAACTTCGTATAAAAATAATTATACGAAGTTAT | 112 |
| ATAACTTCGTATAAAAATATATATACGAAGTTAT | 113 |
| ATAACTTCGTATAAAAATTAATATACGAAGTTAT | 114 |
| ATAACTTCGTATAAAAATTATTATACGAAGTTAT | 115 |
| ATAACTTCGTATAAAAATTTATATACGAAGTTAT | 116 |
| ATAACTTCGTATAAAAATTTTTATACGAAGTTAT | 117 |
| ATAACTTCGTATAAAATAAAATATACGAAGTTAT | 118 |
| ATAACTTCGTATAAAATAAATTATACGAAGTTAT | 119 |
| ATAACTTCGTATAAAATAATATATACGAAGTTAT | 120 |
| ATAACTTCGTATAAAATAATTTATACGAAGTTAT | 121 |
| ATAACTTCGTATAAAATATAATATACGAAGTTAT | 122 |
| ATAACTTCGTATAAAATATATTATACGAAGTTAT | 123 |
| ATAACTTCGTATAAAATATTATATACGAAGTTAT | 124 |
| ATAACTTCGTATAAAATATTTTATACGAAGTTAT | 125 |
| ATAACTTCGTATAAAATTAAATATACGAAGTTAT | 126 |
| ATAACTTCGTATAAAATTAATTATACGAAGTTAT | 127 |
| ATAACTTCGTATAAAAAAAAATATACGAAGTTAT | 128 |
| ATAACTTCGTATAAAATTATTTATACGAAGTTAT | 129 |
| ATAACTTCGTATAAAATTTAATATACGAAGTTAT | 130 |
| ATAACTTCGTATAAAATTTATTATACGAAGTTAT | 131 |
| ATAACTTCGTATAAAATTTTATATACGAAGTTAT | 132 |
| ATAACTTCGTATAAAATTTTTTATACGAAGTTAT | 133 |
| ATAACTTCGTATAAATAAAAATATACGAAGTTAT | 134 |
| ATAACTTCGTATAAATAAAATTATACGAAGTTAT | 135 |
| ATAACTTCGTATAAATAAATATATACGAAGTTAT | 136 |
| ATAACTTCGTATAAATAAATTTATACGAAGTTAT | 137 |
| ATAACTTCGTATAAATAATAATATACGAAGTTAT | 138 |
| ATAACTTCGTATAAATAATATTATACGAAGTTAT | 139 |
| ATAACTTCGTATAAATAATTATATACGAAGTTAT | 140 |
| ATAACTTCGTATAAATAATTTTATACGAAGTTAT | 141 |
| ATAACTTCGTATAAATATAAATATACGAAGTTAT | 142 |
| ATAACTTCGTATAAATATAATTATACGAAGTTAT | 143 |
| ATAACTTCGTATAAATATATATATACGAAGTTAT | 144 |
| ATAACTTCGTATAAATATATTTATACGAAGTTAT | 145 |
| ATAACTTCGTATAAATATTAATATACGAAGTTAT | 146 |
| ATAACTTCGTATAAATATTATTATACGAAGTTAT | 147 |
| ATAACTTCGTATAAATATTTATATACGAAGTTAT | 148 |
| ATAACTTCGTATAAATATTTTTATACGAAGTTAT | 149 |
| ATAACTTCGTATAAATTAAAATATACGAAGTTAT | 150 |
| ATAACTTCGTATAAATTAAATTATACGAAGTTAT | 151 |
| ATAACTTCGTATAAATTAATATATACGAAGTTAT | 152 |
| ATAACTTCGTATAAATTAATTTATACGAAGTTAT | 153 |
| ATAACTTCGTATAAATTATAATATACGAAGTTAT | 154 |
| ATAACTTCGTATAAATTATATTATACGAAGTTAT | 155 |
| ATAACTTCGTATAAATTATTATATACGAAGTTAT | 156 |
| ATAACTTCGTATAAATTATTTTATACGAAGTTAT | 157 |
| ATAACTTCGTATAAATTTAAATATACGAAGTTAT | 158 |
| ATAACTTCGTATAAATTTAATTATACGAAGTTAT | 159 |
| ATAACTTCGTATAAATTTATATATACGAAGTTAT | 160 |
| ATAACTTCGTATAAATTTATTTATACGAAGTTAT | 161 |
| ATAACTTCGTATAAATTTTAATATACGAAGTTAT | 162 |
| ATAACTTCGTATAAATTTTATTATACGAAGTTAT | 163 |
| ATAACTTCGTATAAATTTTTATATACGAAGTTAT | 164 |
| ATAACTTCGTATAAATTTTTTTATACGAAGTTAT | 165 |
| ATAACTTCGTATAATAAAAAATATACGAAGTTAT | 166 |
| ATAACTTCGTATAATAAAAATTATACGAAGTTAT | 167 |
| ATAACTTCGTATAATAAAATATATACGAAGTTAT | 168 |
| ATAACTTCGTATAATAAAATTTATACGAAGTTAT | 169 |
| ATAACTTCGTATAATAAATAATATACGAAGTTAT | 170 |
| ATAACTTCGTATAATAAATATTATACGAAGTTAT | 171 |
| ATAACTTCGTATAATAAATTATATACGAAGTTAT | 172 |
| ATAACTTCGTATAATAAATTTTATACGAAGTTAT | 173 |
| ATAACTTCGTATAATAATAAATATACGAAGTTAT | 174 |
| ATAACTTCGTATAATAATAATTATACGAAGTTAT | 175 |
| ATAACTTCGTATAATAATATATATACGAAGTTAT | 176 |
| ATAACTTCGTATAATAATATTTATACGAAGTTAT | 177 |
| ATAACTTCGTATAATAATTAATATACGAAGTTAT | 178 |
| ATAACTTCGTATAATAATTATTATACGAAGTTAT | 179 |
| ATAACTTCGTATAATAATTTATATACGAAGTTAT | 180 |
| ATAACTTCGTATAATAATTTTTATACGAAGTTAT | 181 |
| ATAACTTCGTATAATATAAAATATACGAAGTTAT | 182 |
| ATAACTTCGTATAATATAAATTATACGAAGTTAT | 183 |
| ATAACTTCGTATAATATAATATATACGAAGTTAT | 184 |
| ATAACTTCGTATAATATAATTTATACGAAGTTAT | 185 |
| ATAACTTCGTATAATATATAATATACGAAGTTAT | 186 |
| ATAACTTCGTATAATATATATTATACGAAGTTAT | 187 |
| ATAACTTCGTATAATATATTATATACGAAGTTAT | 188 |
| ATAACTTCGTATAATATATTTTATACGAAGTTAT | 189 |
| ATAACTTCGTATAATATTAAATATACGAAGTTAT | 190 |
| ATAACTTCGTATAATATTAATTATACGAAGTTAT | 191 |
| ATAACTTCGTATAATATTATATATACGAAGTTAT | 192 |
| ATAACTTCGTATAATATTATTTATACGAAGTTAT | 193 |
| ATAACTTCGTATAATATTTAATATACGAAGTTAT | 194 |
| ATAACTTCGTATAATATTTATTATACGAAGTTAT | 195 |
| ATAACTTCGTATAATATTTTATATACGAAGTTAT | 196 |
| ATAACTTCGTATAATATTTTTTATACGAAGTTAT | 197 |
| ATAACTTCGTATAATTAAAAATATACGAAGTTAT | 198 |
| ATAACTTCGTATAATTAAAATTATACGAAGTTAT | 199 |
| ATAACTTCGTATAATTAAATATATACGAAGTTAT | 200 |
| ATAACTTCGTATAATTAAATTTATACGAAGTTAT | 201 |
| ATAACTTCGTATAATTAATAATATACGAAGTTAT | 202 |
| ATAACTTCGTATAATTAATATTATACGAAGTTAT | 203 |
| ATAACTTCGTATAATTAATTATATACGAAGTTAT | 204 |
| ATAACTTCGTATAATTAATTTTATACGAAGTTAT | 205 |
| ATAACTTCGTATAATTATAAATATACGAAGTTAT | 206 |
| ATAACTTCGTATAATTATAATTATACGAAGTTAT | 207 |
| ATAACTTCGTATAATTATATATATACGAAGTTAT | 208 |
| ATAACTTCGTATAATTATATTTATACGAAGTTAT | 209 |
| ATAACTTCGTATAATTATTAATATACGAAGTTAT | 210 |
| ATAACTTCGTATAATTATTATTATACGAAGTTAT | 211 |
| ATAACTTCGTATAATTATTTATATACGAAGTTAT | 212 |
| ATAACTTCGTATAATTATTTTTATACGAAGTTAT | 213 |
| ATAACTTCGTATAATTTAAAATATACGAAGTTAT | 214 |
| ATAACTTCGTATAATTTAAATTATACGAAGTTAT | 215 |
| ATAACTTCGTATAATTTAATATATACGAAGTTAT | 216 |
| ATAACTTCGTATAATTTAATTTATACGAAGTTAT | 217 |
| ATAACTTCGTATAATTTATAATATACGAAGTTAT | 218 |
| ATAACTTCGTATAATTTATATTATACGAAGTTAT | 219 |
| ATAACTTCGTATAATTTATTATATACGAAGTTAT | 220 |
| ATAACTTCGTATAATTTATTTTATACGAAGTTAT | 221 |
| ATAACTTCGTATAATTTTAAATATACGAAGTTAT | 222 |
| ATAACTTCGTATAATTTTAATTATACGAAGTTAT | 223 |
| ATAACTTCGTATAATTTTATATATACGAAGTTAT | 224 |
| ATAACTTCGTATAATTTTATTTATACGAAGTTAT | 225 |
| ATAACTTCGTATAATTTTTAATATACGAAGTTAT | 226 |
| ATAACTTCGTATAATTTTTATTATACGAAGTTAT | 227 |
| ATAACTTCGTATAATTTTTTATATACGAAGTTAT | 228 |
| ATAACTTCGTATAATTTTTTTTATACGAAGTTAT | 229 |
| ATAACTTCGTATATAAAAAAATATACGAAGTTAT | 230 |
| ATAACTTCGTATATAAAAAATTATACGAAGTTAT | 231 |
| ATAACTTCGTATATAAAAATATATACGAAGTTAT | 232 |
| ATAACTTCGTATATAAAAATTTATACGAAGTTAT | 233 |
| ATAACTTCGTATATAAAATAATATACGAAGTTAT | 234 |
| ATAACTTCGTATATAAAATATTATACGAAGTTAT | 235 |
| ATAACTTCGTATATAAAATTATATACGAAGTTAT | 236 |
| ATAACTTCGTATATAAAATTTTATACGAAGTTAT | 237 |
| ATAACTTCGTATATAAATAAATATACGAAGTTAT | 238 |
| ATAACTTCGTATATAAATAATTATACGAAGTTAT | 239 |
| ATAACTTCGTATATAAATATATATACGAAGTTAT | 240 |
| ATAACTTCGTATATAAATATTTATACGAAGTTAT | 241 |
| ATAACTTCGTATATAAATTAATATACGAAGTTAT | 242 |
| ATAACTTCGTATATAAATTATTATACGAAGTTAT | 243 |
| ATAACTTCGTATATAAATTTATATACGAAGTTAT | 244 |
| ATAACTTCGTATATAAATTTTTATACGAAGTTAT | 245 |
| ATAACTTCGTATATAATAAAATATACGAAGTTAT | 246 |
| ATAACTTCGTATATAATAAATTATACGAAGTTAT | 247 |
| ATAACTTCGTATATAATAATATATACGAAGTTAT | 248 |
| ATAACTTCGTATATAATAATTTATACGAAGTTAT | 249 |
| ATAACTTCGTATATAATATAATATACGAAGTTAT | 250 |
| ATAACTTCGTATATAATATATTATACGAAGTTAT | 251 |
| ATAACTTCGTATATAATATTATATACGAAGTTAT | 252 |
| ATAACTTCGTATATAATATTTTATACGAAGTTAT | 253 |
| ATAACTTCGTATATAATTAAATATACGAAGTTAT | 254 |
| ATAACTTCGTATATAATTAATTATACGAAGTTAT | 255 |
| ATAACTTCGTATATAATTATATATACGAAGTTAT | 256 |
| ATAACTTCGTATATAATTATTTATACGAAGTTAT | 257 |
| ATAACTTCGTATATAATTTAATATACGAAGTTAT | 258 |
| ATAACTTCGTATATAATTTATTATACGAAGTTAT | 259 |
| ATAACTTCGTATATAATTTTATATACGAAGTTAT | 260 |
| ATAACTTCGTATATAATTTTTTATACGAAGTTAT | 261 |
| ATAACTTCGTATATATAAAAATATACGAAGTTAT | 262 |
| ATAACTTCGTATATATAAAATTATACGAAGTTAT | 263 |
| ATAACTTCGTATATATAAATATATACGAAGTTAT | 264 |
| ATAACTTCGTATATATAAATTTATACGAAGTTAT | 265 |
| ATAACTTCGTATATATAATAATATACGAAGTTAT | 266 |
| ATAACTTCGTATATATAATATTATACGAAGTTAT | 267 |
| ATAACTTCGTATATATAATTATATACGAAGTTAT | 268 |
| ATAACTTCGTATATATAATTTTATACGAAGTTAT | 269 |
| ATAACTTCGTATATATATAAATATACGAAGTTAT | 270 |
| ATAACTTCGTATATATATAATTATACGAAGTTAT | 271 |
| ATAACTTCGTATATATATATATATACGAAGTTAT | 272 |
| ATAACTTCGTATATATATATTTATACGAAGTTAT | 273 |
| ATAACTTCGTATATATATTAATATACGAAGTTAT | 274 |
| ATAACTTCGTATATATATTATTATACGAAGTTAT | 275 |
| ATAACTTCGTATATATATTTATATACGAAGTTAT | 276 |
| ATAACTTCGTATATATATTTTTATACGAAGTTAT | 277 |
| ATAACTTCGTATATATTAAAATATACGAAGTTAT | 278 |
| ATAACTTCGTATATATTAAATTATACGAAGTTAT | 279 |
| ATAACTTCGTATATATTAATATATACGAAGTTAT | 280 |
| ATAACTTCGTATATATTAATTTATACGAAGTTAT | 281 |
| ATAACTTCGTATATATTATAATATACGAAGTTAT | 282 |
| ATAACTTCGTATATATTATATTATACGAAGTTAT | 283 |
| ATAACTTCGTATATATTATTATATACGAAGTTAT | 284 |
| ATAACTTCGTATATATTATTTTATACGAAGTTAT | 285 |
| ATAACTTCGTATATATTTAAATATACGAAGTTAT | 286 |
| ATAACTTCGTATATATTTAATTATACGAAGTTAT | 287 |
| ATAACTTCGTATATATTTATATATACGAAGTTAT | 288 |
| ATAACTTCGTATATATTTATTTATACGAAGTTAT | 289 |
| ATAACTTCGTATATATTTTAATATACGAAGTTAT | 290 |
| ATAACTTCGTATATATTTTATTATACGAAGTTAT | 291 |
| ATAACTTCGTATATATTTTTATATACGAAGTTAT | 292 |
| ATAACTTCGTATATATTTTTTTATACGAAGTTAT | 293 |
| ATAACTTCGTATATTAAAAAATATACGAAGTTAT | 294 |
| ATAACTTCGTATATTAAAAATTATACGAAGTTAT | 295 |
| ATAACTTCGTATATTAAAATATATACGAAGTTAT | 296 |
| ATAACTTCGTATATTAAAATTTATACGAAGTTAT | 297 |
| ATAACTTCGTATATTAAATAATATACGAAGTTAT | 298 |
| ATAACTTCGTATATTAAATATTATACGAAGTTAT | 299 |
| ATAACTTCGTATATTAAATTATATACGAAGTTAT | 300 |
| ATAACTTCGTATATTAAATTTTATACGAAGTTAT | 301 |
| ATAACTTCGTATATTAATAAATATACGAAGTTAT | 302 |
| ATAACTTCGTATATTAATAATTATACGAAGTTAT | 303 |
| ATAACTTCGTATATTAATATATATACGAAGTTAT | 304 |
| ATAACTTCGTATATTAATATTTATACGAAGTTAT | 305 |
| ATAACTTCGTATATTAATTAATATACGAAGTTAT | 306 |
| ATAACTTCGTATATTAATTATTATACGAAGTTAT | 307 |
| ATAACTTCGTATATTAATTTATATACGAAGTTAT | 308 |
| ATAACTTCGTATATTAATTTTTATACGAAGTTAT | 309 |
| ATAACTTCGTATATTATAAAATATACGAAGTTAT | 310 |
| ATAACTTCGTATATTATAAATTATACGAAGTTAT | 311 |
| ATAACTTCGTATATTATAATATATACGAAGTTAT | 312 |
| ATAACTTCGTATATTATAATTTATACGAAGTTAT | 313 |
| ATAACTTCGTATATTATATAATATACGAAGTTAT | 314 |
| ATAACTTCGTATATTATATATTATACGAAGTTAT | 315 |
| ATAACTTCGTATATTATATTATATACGAAGTTAT | 316 |
| ATAACTTCGTATATTATATTTTATACGAAGTTAT | 317 |
| ATAACTTCGTATATTATTAAATATACGAAGTTAT | 318 |
| ATAACTTCGTATATTATTAATTATACGAAGTTAT | 319 |
| ATAACTTCGTATATTATTATATATACGAAGTTAT | 320 |
| ATAACTTCGTATATTATTATTTATACGAAGTTAT | 321 |
| ATAACTTCGTATATTATTTAATATACGAAGTTAT | 322 |
| ATAACTTCGTATATTATTTATTATACGAAGTTAT | 323 |
| ATAACTTCGTATATTATTTTATATACGAAGTTAT | 324 |
| ATAACTTCGTATATTATTTTTTATACGAAGTTAT | 325 |
| ATAACTTCGTATATTTAAAAATATACGAAGTTAT | 326 |
| ATAACTTCGTATATTTAAAATTATACGAAGTTAT | 327 |
| ATAACTTCGTATATTTAAATATATACGAAGTTAT | 328 |
| ATAACTTCGTATATTTAAATTTATACGAAGTTAT | 329 |
| ATAACTTCGTATATTTAATAATATACGAAGTTAT | 330 |
| ATAACTTCGTATATTTAATATTATACGAAGTTAT | 331 |
| ATAACTTCGTATATTTAATTATATACGAAGTTAT | 332 |
| ATAACTTCGTATATTTAATTTTATACGAAGTTAT | 333 |
| ATAACTTCGTATATTTATAAATATACGAAGTTAT | 334 |
| ATAACTTCGTATATTTATAATTATACGAAGTTAT | 335 |
| ATAACTTCGTATATTTATATATATACGAAGTTAT | 336 |
| ATAACTTCGTATATTTATATTTATACGAAGTTAT | 337 |
| ATAACTTCGTATATTTATTAATATACGAAGTTAT | 338 |
| ATAACTTCGTATATTTATTATTATACGAAGTTAT | 339 |
| ATAACTTCGTATATTTATTTATATACGAAGTTAT | 340 |
| ATAACTTCGTATATTTATTTTTATACGAAGTTAT | 341 |
| ATAACTTCGTATATTTTAAAATATACGAAGTTAT | 342 |
| ATAACTTCGTATATTTTAAATTATACGAAGTTAT | 343 |
| ATAACTTCGTATATTTTAATATATACGAAGTTAT | 344 |
| ATAACTTCGTATATTTTAATTTATACGAAGTTAT | 345 |
| ATAACTTCGTATATTTTATAATATACGAAGTTAT | 346 |
| ATAACTTCGTATATTTTATATTATACGAAGTTAT | 347 |
| ATAACTTCGTATATTTTATTATATACGAAGTTAT | 348 |
| ATAACTTCGTATATTTTATTTTATACGAAGTTAT | 349 |
| ATAACTTCGTATATTTTTAAATATACGAAGTTAT | 350 |
| ATAACTTCGTATATTTTTAATTATACGAAGTTAT | 351 |
| ATAACTTCGTATATTTTTATATATACGAAGTTAT | 352 |
| ATAACTTCGTATATTTTTATTTATACGAAGTTAT | 353 |
| ATAACTTCGTATATTTTTTAATATACGAAGTTAT | 354 |
| ATAACTTCGTATATTTTTTATTATACGAAGTTAT | 355 |
| ATAACTTCGTATATTTTTTTATATACGAAGTTAT | 356 |
| ATAACTTCGTATATTTTTTTTTATACGAAGTTAT | 357 |

In certain specific embodiments that LoxP arm 1 (SEQ ID NO:1) and LoxParm 2 (SEQ ID NO:2) have been combined with the core spacer sequences designated as loxW1 to loxW12, loxW15 to loxW26, and loxW28 above and have validated in the screening methods provided herein.

### EXAMPLES

### Example 1—Screening Method for Methylation Resistant Lox Sites

Example one describes a screening method used for determining the nucleotide sequences for methylation resistant lox sites. Vectors were constructed having flanking degenerate primer sequences (both 3′ and 5′) whose overhangs included LoxP sites with loxP arm 1 (SEQ ID NO:1), a core spacer sequence which only contained the DNA bases A and T, and loxP arm 2 (SEQ ID NO:2) for use in PCR amplification of the linear product containing a bacterial origin of replication sequence and an antibiotic resistance gene (AmpR). (FIG. 2). The PCR products were treated in vitro with purified Cre recombinase, and those vectors with compatible lox sites with cytosine free core spacer sequences were circularized into plasmids capable of replication and selection in E. coli. (FIG. 2, left panel). Colonies produced through this method were sequenced. (FIG. 2, upper right panel). The resulting lox sites with cytosine-free core spacer sequences were screened for activity through cloning two direct repeats in plasmids and treating with purified Cre recombinase for excision products. (FIG. 2, lower right panel). Lox core spacer sequences loxW1 to loxW12, loxW15 to loxW26, and loxW28 described and disclosed above and have been fully validated using this method.

### Example 2—Test for Increased Recombination in Plant Cells

To implement the discovered lox sites and to test for increased recombination in plant cells, well-performing sites were cloned by standard methods into a vector in direct repeats, the first site was placed in the 5′ UTR of a reporter gene (the fluorescent reporter tdTomato), and the second site was placed after the terminator of the same fluorescent protein gene. A second fluorescent reporter (green fluorescent protein, GFP) was placed downstream of this second lox site without a promoter such that excision via Cre recombinase of the first product turns on expression of the second reporter. Testing of these vectors with a constitutively expressed Cre recombinase in tobacco in well-known agrofiltration assays showed significantly increased transgene excision with loxP cytosine-free core spacer sequences relative to the same vector with traditional wild-type loxP sites as shown in FIG. 3.

In a second test, the vectors were transformed into hybrid poplar (Populus tremula x alba) by standard treatment with Agrobacterium. Vectors with LoxP and Lox W7-containing transgenes with constitutive Cre expression and a methylation-sensitive excision reporter (FIG. 4A) exhibited reduced shoot regeneration rates versus no Cre containing control vectors in poplar. Vectors with the LoxW7 transgene allow for approximately 10% excised shoot recovery. (FIG. 4A).

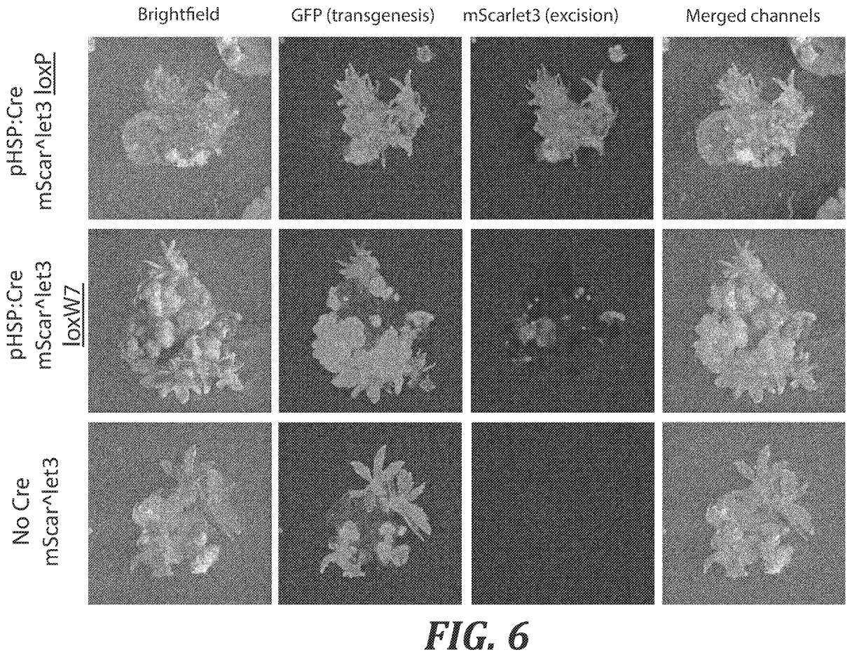

A second vector configuration was also tested in poplar. In the second vector configuration, a fluorescent reporter coding sequence (mScarlet3) was separated into two exons with a third exon in the middle of the gene encoding an inflexible linker. The inflexible linker prevented proper folding of the fluorescent protein. (FIG. 4B). Because the sequence contexts of each of the two vector constructs around the lox sites have very different GC content and important gene features, the first promoter-less switch reporter was anticipated to be DNA-methylation sensitive, and the latter intron-linker excision reporter (which has 26% GC content in introns around lox sequences) to be DNA-methylation resistant. Testing of the DNA methylation-sensitive reporter with a constitutively expressed Cre in tobacco agroinfiltration assays showed significantly increased transgene excision with cytosine-free spacer lox sites relative to the same vector with traditional wild-type loxP sites (FIG. 3). The same result was found in stable transgenic calli and shoots in the hybrid poplar P. tremula x alba using the DNA methylation-sensitive reporter and constitutive Cre recombinase (FIG. 4B). Using the DNA methylation-resistant reporter and constitutive Cre recombinase, cytosine-free core spacer sequences resulted in greater excised plant recovery compared with loxP and reduced negative impacts on plant regeneration (FIG. 4A). With a heat-shock inducible Cre recombinase coupled with a methylation-resistant reporter, loxP containing transgenic poplars excised uncontrollably without heat-shock stimulus, whereas in the lox site with a cytosine-free core spacer sequence containing transgenic poplars, shoots remained unexcised (FIG. 6). This result additionally suggested that lox sites with cytosine-free core spacer sequences have enhanced dynamic control of excision compared with conventional loxP sites.

### Example 3—Use of Lox Sites with a Cystosine-free Core Spacer Sequence for Excision of a Morphogenic Gene and CRISPR/Cas9 Transgenes

Example 3 provides an example of the use of lox sites with a cytosine-free core spacer sequence. T-DNA vectors which contain morphogenic root inducing genes (rol genes from Agrobacterium rhizogenes strain A4), morphogenic shoot-inducing genes (WUSCHEL (WUS) from Arabidopsis thaliana and ipt from Agrobacterium tumefasciens), as well as fluorescent markers, a red betalain pigment gene (RUBY), Cre recombinase, Cas9, and gRNAs was assembled and flanked by cytosine-free lox sites (FIG. 7A). This assembly was produced using cloning techniques common to those skilled in the art. After transformation and recovery of transgenic roots, shoot formation and transgene excision were induced by heat shock for two weeks at 38° C. for four hours daily, and many shoots lacking transgenic markers were found and propagated successfully (FIG. 7B). When analyzed in detail, 48% of recovered initial transgenic root events could form excised shoots lacking marker gene expression, and 50% of surveyed product shoots were fully excised of transgene components at the DNA level (FIG. 7C). These vectors show highly efficient generation of transgene-excised plants using lox sites having cytosine-free core spacer sequences.

While representative embodiments have been illustrated and described, it will be appreciated that various changes can be made therein without departing from the spirit and scope of the compositions and methods provided and claimed.

1. A plant lox site comprising a lox arm one and a lox arm two separated by a cytosine free core spacer sequence, wherein the core spacer sequence comprises between four and eight randomly repeating adenine and thymine nucleotides.

2. The plant lox site according to claim 1, wherein the core spacer sequence comprises eight nucleotides.

3. The plant lox site according to claim 1, wherein each of lox arm one and lox arm two independently comprises a nucleotide sequence SEQ ID NO:1, SEQ ID NO:2, or SEQ ID NO:4 through SEQ ID NO: 102.

4. (canceled)

5. The plant lox site according to claim 1, wherein the core spacer sequence is one of AAAAAAAA, AAAAAAAT, AAAAAATA, AAAAAATT, AAAAATAA, AAAAATAT, AAAAATTA, AAAAATTT, AAAATAAA, AAAATAAT, AAAATATA, AAAATATT, AAAATTAA, AAAATTAT, AAAATTTA, AAAATTTT, AAATAAAA, AAATAAAT, AAATAATA, AAATAATT, AAATATAA, AAATATAT, AAATATTA, AAATATTT, AAATTAAA, AAATTAAT, AAATTATA, AAATTATT, AAATTTAA, AAATTTAT, AAATTTTA, AAATTTTT, AATAAAAA, AATAAAAT, AATAAATA, AATAAATT, AATAATAA, AATAATAT, AATAATTA, AATAATTT, AATATAAA, AATATAAT, AATATATA, AATATATT, AATATTAA, AATATTAT, AATATTTA, AATATTTT, AATTAAAA, AATTAAAT, AATTAATA, AATTAATT, AATTATAA, AATTATAT, AATTATTA, AATTATTT, AATTTAAA, AATTTAAT, AATTTATA, AATTTATT, AATTTTAA, AATTTTAT, AATTTTTA, AATTTTTT, ATAAAAAA, ATAAAAAT, ATAAAATA, ATAAAATT, ATAAATAA, ATAAATAT, ATAAATTA, ATAAATTT, ATAATAAA, ATAATAAT, ATAATATA, ATAATATT, ATAATTAA, ATAATTAT, ATAATTTA, ATAATTTT, ATATAAAA, ATATAAAT, ATATAATA, ATATAATT, ATATATAA, ATTTAATA, ATATATAT, ATATATTA, ATATATTT, ATATTAAA, ATATTAAT, ATATTATA, ATATTATT, ATATTTAA, ATATTTAT, ATATTTTA, ATATTTTT, ATTAAAAA, ATTAAAAT, ATTAAATA, ATTAAATT, ATTAATAA, ATTAATAT, ATTAATTA, ATTAATTT, ATTATAAA, ATTATAAT, ATTATATA, ATTATATT, ATTATTAA, ATTATTAT, ATTATTTA, ATTATTTT, ATTTAAAA, ATTTAAAT, TAAATTTT, ATTTAATT, ATTTATAA, ATTTATAT, ATTTATTA, ATTTATTT, ATTTTAAA, ATTTTAAT, ATTTTATA, ATTTTATT, ATTTTTAA, ATTTTTAT, ATTTTTTA, ATTTTTTT, TAAAAAAA, TAAAAAAT, TAAAAATA, TAAAAATT, TAAAATAA, TAAAATAT, TAAAATTA, TAAAATTT, TAAATAAA, TAAATAAT, TAAATATA, TAAATATT, TAAATTAA, TAAATTAT, TAAATTTA, TATATTAA, TATATTAT, TAATAAAA, TAATAAAT, TAATAATA, TAATAATT, TAATATAA, TAATATAT, TAATATTA, TAATATTT, TAATTAAA, TAATTAAT, TAATTATA, TAATTATT, TAATTTAA, TAATTTAT, TAATTTTA, TAATTTTT, TATAAAAA, TATAAAAT, TATAAATA, TATAAATT, TATAATAA, TATAATAT, TATAATTA, TATAATTT, TATATAAA, TATATAAT, TATATATA, TATATATT, TTAATAAT, TTAATATA, TATATTTA, TATATTTT, TATTAAAA, TATTAAAT, TATTAATA, TATTAATT, TATTATAA, TATTATAT, TATTATTA, TATTATTT, TATTTAAA, TATTTAAT, TATTTATA, TATTTATT, TATTTTAA, TATTTTAT, TATTTTTA, TATTTTTT, TTAAAAAA, TTAAAAAT, TTAAAATA, TTAAAATT, TTAAATAA, TTAAATAT, TTAAATTA, TTAAATTT, TTAATAAA, TTTAATTA, TTTAATTT, TTTATAAA, TTAATATT, TTAATTAA, TTAATTAT, TTAATTTA, TTAATTTT, TTATAAAA, TTATAAAT, TTATAATA, TTATAATT, TTATATAA, TTATATAT, TTATATTA, TTATATTT, TTATTAAA, TTATTAAT, TTATTATA, TTATTATT, TTATTTAA, TTATTTAT, TTATTTTA, TTATTTTT, TTTAAAAA, TTTAAAAT, TTTAAATA, TTTAAATT, TTTAATAA, TTTAATAT, TTTTAATT, TTTTATAA, TTTTATAT, TTTATAAT, TTTATATA, TTTATATT, TTTATTAA, TTTATTAT, TTTATTTA, TTTATTTT, TTTTAAAA, TTTTAAAT, TTTTAATA, TTTTATTA, TTTTATTT, TTTTTAAA, TTTTTAAT, TTTTTATA, TTTTTATT, TTTTTTAA, TTTTTTAT, TTTTTTTA, or TTTTTTTT.

6. The plant lox site according to claim 1, wherein the plant lox site comprises one of SEQ ID NO: 103-SEQ ID NO:357.

7. A method to increase the efficiency of Cre-mediated transgene excision in a plant cell, the method comprising:
(a) introducing into at least one plant cell at least one plasmid comprising at least one plant lox site according to claim 1; and
(b) contacting the at least one plant lox site with a Cre recombinase,
wherein the transgene is excised more efficiently when the at least one plant lox site is contacted with the Cre recombinase compared to transgene excision in the absence of the at least one plant lox site.

8. The method according to claim 7, wherein the plasmid comprises a transgene flanked by direct repeats of the plant lox site.

9. The method according to claim 8, wherein the transgene comprises Cas 9 and at least one guide RNA.

10. The method according to claim 7, wherein the plasmid further comprises a Cre coding region.

11. The method according to claim 7, wherein the plant lox site is comprises any one of SEQ ID NO:103-SEQ ID NO:357.

12. The method according to claim 7, wherein the plasmid comprises a constitutive promoter, an inducible promoter, or a tissue specific promoter controlling Cre expression.

13. The method according to claim 7, wherein the plasmid comprises a reporter gene.

14. The method according to claim 13, wherein the reporter is a fluorescent or DNA methylation sensitive reporter.

15. A plasmid or vector comprising the plant lox site according to claim 1 wherein the transgene is flanked by the direct repeats of the plant lox site.

16. A plasmid or vector comprising the plant lox site according to claim 2 wherein the transgene is flanked by the direct repeats of the plant lox site.

17. A plasmid or vector comprising the plant lox site according to claim 3 wherein the transgene is flanked by the direct repeats of the plant lox site.

18. A plasmid or vector comprising the plant lox site according to claim 4 wherein the transgene is flanked by the direct repeats of the plant lox site.

19. A plasmid or vector comprising the plant lox site according to claim 5 wherein the transgene is flanked by the direct repeats of the plant lox site.

20. A plasmid or vector comprising the plant lox site according to claim 6 wherein the transgene is flanked by the direct repeats of the plant lox site.


SEQUENCE LISTING (357 sequences)

SEQ ID NO: 1 - DNA, 13 bp, P1 bacteriophage
  source 1..13: mol_type = other DNA
ataacttcgtata

SEQ ID NO: 2 - DNA, 13 bp, P1 bacteriophage
  source 1..13: mol_type = other DNA
tatacgaagttat

SEQ ID NO: 3 - DNA, 13 bp, synthetic construct
  source 1..13: mol_type = other DNA
actatgtcgcact

SEQ ID NO: 4 - DNA, 13 bp, synthetic construct
  source 1..13: mol_type = other DNA
agtgcgacatagt

SEQ ID NO: 5 - DNA, 13 bp, synthetic construct
  source 1..13: mol_type = other DNA
gaaaggggctata

SEQ ID NO: 6 - DNA, 13 bp, synthetic construct
  source 1..13: mol_type = other DNA
tatagcccctttc

SEQ ID NO: 7 - DNA, 13 bp, synthetic construct
  source 1..13: mol_type = other DNA
ctgcaatctcatt

SEQ ID NO: 8 - DNA, 13 bp, synthetic construct
  source 1..13: mol_type = other DNA
aatgagattgcag

SEQ ID NO: 9 - DNA, 13 bp, synthetic construct
  source 1..13: mol_type = other DNA
gcaccagcctatc

SEQ ID NO: 10 - DNA, 13 bp, synthetic construct
  source 1..13: mol_type = other DNA
gataggctggtgc

SEQ ID NO: 11 - DNA, 13 bp, synthetic construct
  source 1..13: mol_type = other DNA
caaaatccggaaa

SEQ ID NO: 12 - DNA, 13 bp, synthetic construct
  source 1..13: mol_type = other DNA
tttccggattttg

SEQ ID NO: 13 - DNA, 13 bp, synthetic construct
  source 1..13: mol_type = other DNA
ctagccacaataa

SEQ ID NO: 14 - DNA, 13 bp, synthetic construct
  source 1..13: mol_type = other DNA
ttattgtggctag

SEQ ID NO: 15 - DNA, 13 bp, synthetic construct
  source 1..13: mol_type = other DNA
acagaaacgcgtt

SEQ ID NO: 16 - DNA, 13 bp, synthetic construct
  source 1..13: mol_type = other DNA
aacgcgtttctgt

SEQ ID NO: 17 - DNA, 13 bp, synthetic construct
  source 1..13: mol_type = other DNA
aacaagtcatata

SEQ ID NO: 18 - DNA, 13 bp, synthetic construct
  source 1..13: mol_type = other DNA
tatatgacttgtt

SEQ ID NO: 19 - DNA, 13 bp, synthetic construct
  source 1..13: mol_type = other DNA
caagtttcaataa

SEQ ID NO: 20 - DNA, 13 bp, synthetic construct
  source 1..13: mol_type = other DNA
ttattgaaacttg

SEQ ID NO: 21 - DNA, 13 bp, synthetic construct
  source 1..13: mol_type = other DNA
aaccgatcccctc

SEQ ID NO: 22 - DNA, 13 bp, synthetic construct
  source 1..13: mol_type = other DNA
gaggggatcggtt

SEQ ID NO: 23 - DNA, 13 bp, synthetic construct
  source 1..13: mol_type = other DNA
aaaacccccataa

SEQ ID NO: 24 - DNA, 13 bp, synthetic construct
  source 1..13: mol_type = other DNA
ttatgggggtttt

SEQ ID NO: 25 - DNA, 13 bp, synthetic construct
  source 1..13: mol_type = other DNA
cactggccgtatc

SEQ ID NO: 26 - DNA, 13 bp, synthetic construct
  source 1..13: mol_type = other DNA
gatacggccagtg

SEQ ID NO: 27 - DNA, 13 bp, synthetic construct
  source 1..13: mol_type = other DNA
ttacttgaaccac

SEQ ID NO: 28 - DNA, 13 bp, synthetic construct
  source 1..13: mol_type = other DNA
gtggttcaagtaa

SEQ ID NO: 29 - DNA, 13 bp, synthetic construct
  source 1..13: mol_type = other DNA
caccatgtgtatt

SEQ ID NO: 30 - DNA, 13 bp, synthetic construct
  source 1..13: mol_type = other DNA
aatacacatggtg

SEQ ID NO: 31 - DNA, 13 bp, synthetic construct
  source 1..13: mol_type = other DNA
tggatccgaaata

SEQ ID NO: 32 - DNA, 13 bp, synthetic construct
  source 1..13: mol_type = other DNA
tatttcggatcca

SEQ ID NO: 33 - DNA, 13 bp, synthetic construct
  source 1..13: mol_type = other DNA
atcacgatgactt

SEQ ID NO: 34 - DNA, 13 bp, synthetic construct
  source 1..13: mol_type = other DNA
aagtcatcgtgat

SEQ ID NO: 35 - DNA, 13 bp, synthetic construct
  source 1..13: mol_type = other DNA
aatatatcaactg

SEQ ID NO: 36 - DNA, 13 bp, synthetic construct
  source 1..13: mol_type = other DNA
cagttgatatatt

SEQ ID NO: 37 - DNA, 13 bp, synthetic construct
  source 1..13: mol_type = other DNA
aaacacgccaaca

SEQ ID NO: 38 - DNA, 13 bp, synthetic construct
  source 1..13: mol_type = other DNA
tgttggcgtgttt

SEQ ID NO: 39 - DNA, 13 bp, synthetic construct
  source 1..13: mol_type = other DNA
ttgccacggctaa

SEQ ID NO: 40 - DNA, 13 bp, synthetic construct
  source 1..13: mol_type = other DNA
ttagccgtggcaa

SEQ ID NO: 41 - DNA, 13 bp, synthetic construct
  source 1..13: mol_type = other DNA
ctcactaccaaaa

SEQ ID NO: 42 - DNA, 13 bp, synthetic construct
  source 1..13: mol_type = other DNA
ttttggtagtgag

SEQ ID NO: 43 - DNA, 13 bp, synthetic construct
  source 1..13: mol_type = other DNA
ataacacaccaat

SEQ ID NO: 44 - DNA, 13 bp, synthetic construct
  source 1..13: mol_type = other DNA
attggtgtgttat

SEQ ID NO: 45 - DNA, 13 bp, synthetic construct
  source 1..13: mol_type = other DNA
aacaccaccgata

SEQ ID NO: 46 - DNA, 13 bp, synthetic construct
  source 1..13: mol_type = other DNA
tatcggtggtgtt

SEQ ID NO: 47 - DNA, 13 bp, synthetic construct
  source 1..13: mol_type = other DNA
aagcaagccatta

SEQ ID NO: 48 - DNA, 13 bp, synthetic construct
  source 1..13: mol_type = other DNA
taatggcttgctt

SEQ ID NO: 49 - DNA, 13 bp, synthetic construct
  source 1..13: mol_type = other DNA
caacacaaaaata

SEQ ID NO: 50 - DNA, 13 bp, synthetic construct
  source 1..13: mol_type = other DNA
tatttttgtgttg

SEQ ID NO: 51 - DNA, 13 bp, synthetic construct
  source 1..13: mol_type = other DNA
tctccacacatag

SEQ ID NO: 52 - DNA, 13 bp, synthetic construct
  source 1..13: mol_type = other DNA
ctatgtgtggaga

SEQ ID NO: 53 - DNA, 13 bp, synthetic construct
  source 1..13: mol_type = other DNA
taaaaacgacata

SEQ ID NO: 54 - DNA, 13 bp, synthetic construct
  source 1..13: mol_type = other DNA
tatgtcgttttta

SEQ ID NO: 55 - DNA, 13 bp, synthetic construct
  source 1..13: mol_type = other DNA
ataaacccactca

SEQ ID NO: 56 - DNA, 13 bp, synthetic construct
  source 1..13: mol_type = other DNA
tgagtgggtttat

SEQ ID NO: 57 - DNA, 13 bp, synthetic construct
  source 1..13: mol_type = other DNA
atcgcagtctacg

SEQ ID NO: 58 - DNA, 13 bp, synthetic construct
  source 1..13: mol_type = other DNA
cgtagactgcgat

SEQ ID NO: 59 - DNA, 13 bp, synthetic construct
  source 1..13: mol_type = other DNA
ctaaggacgcata

SEQ ID NO: 60 - DNA, 13 bp, synthetic construct
  source 1..13: mol_type = other DNA
tatgcgtccttag

SEQ ID NO: 61 - DNA, 13 bp, synthetic construct
  source 1..13: mol_type = other DNA
atgggcccagata

SEQ ID NO: 62 - DNA, 13 bp, synthetic construct
  source 1..13: mol_type = other DNA
tatctgggcccat

SEQ ID NO: 63 - DNA, 13 bp, synthetic construct
  source 1..13: mol_type = other DNA
acaacctccctta

SEQ ID NO: 64 - DNA, 13 bp, synthetic construct
  source 1..13: mol_type = other DNA
taagggaggttgt

SEQ ID NO: 65 - DNA, 13 bp, synthetic construct
  source 1..13: mol_type = other DNA
caaacacccggca

SEQ ID NO: 66 - DNA, 13 bp, synthetic construct
  source 1..13: mol_type = other DNA
tgccgggtgtttg

SEQ ID NO: 67 - DNA, 13 bp, synthetic construct
  source 1..13: mol_type = other DNA
ccacctacagaga

SEQ ID NO: 68 - DNA, 13 bp, synthetic construct
  source 1..13: mol_type = other DNA
tctctgtaggtgg

SEQ ID NO: 69 - DNA, 13 bp, synthetic construct
  source 1..13: mol_type = other DNA
tgaacgcaagaaa

SEQ ID NO: 70 - DNA, 13 bp, synthetic construct
  source 1..13: mol_type = other DNA
tttcttgcgttca

SEQ ID NO: 71 - DNA, 13 bp, synthetic construct
  source 1..13: mol_type = other DNA
atacttacacaat

SEQ ID NO: 72 - DNA, 13 bp, synthetic construct
  source 1..13: mol_type = other DNA
attgtgtaagtat

SEQ ID NO: 73 - DNA, 13 bp, synthetic construct
  source 1..13: mol_type = other DNA
aaacaatcccatg

SEQ ID NO: 74 - DNA, 13 bp, synthetic construct
  source 1..13: mol_type = other DNA
catgggattgttt

SEQ ID NO: 75 - DNA, 13 bp, synthetic construct
  source 1..13: mol_type = other DNA
acagcctcggagt

SEQ ID NO: 76 - DNA, 13 bp, synthetic construct
  source 1..13: mol_type = other DNA
actccgaggctgt

SEQ ID NO: 77 - DNA, 13 bp, synthetic construct
  source 1..13: mol_type = other DNA
aaagctcctacta

SEQ ID NO: 78 - DNA, 13 bp, synthetic construct
  source 1..13: mol_type = other DNA
tagtaggagcttt

SEQ ID NO: 79 - DNA, 13 bp, synthetic construct
  source 1..13: mol_type = other DNA
aaccacagtataa

SEQ ID NO: 80 - DNA, 13 bp, synthetic construct
  source 1..13: mol_type = other DNA
ttatactgtggtt

SEQ ID NO: 81 - DNA, 13 bp, synthetic construct
  source 1..13: mol_type = other DNA
aaagcgcccggta

SEQ ID NO: 82 - DNA, 13 bp, synthetic construct
  source 1..13: mol_type = other DNA
taccgggcgcttt

SEQ ID NO: 83 - DNA, 13 bp, synthetic construct
  source 1..13: mol_type = other DNA
ttagatagcgaga

SEQ ID NO: 84 - DNA, 13 bp, synthetic construct
  source 1..13: mol_type = other DNA
tctcgctatctaa

SEQ ID NO: 85 - DNA, 13 bp, synthetic construct
  source 1..13: mol_type = other DNA
ataacttcatata

SEQ ID NO: 86 - DNA, 13 bp, synthetic construct
  source 1..13: mol_type = other DNA
tatatgaagttat

SEQ ID NO: 87 - DNA, 13 bp, synthetic construct
  source 1..13: mol_type = other DNA
ataacttcgtaca

SEQ ID NO: 88 - DNA, 13 bp, synthetic construct
  source 1..13: mol_type = other DNA
tgtacgaagttat

SEQ ID NO: 89 - DNA, 13 bp, synthetic construct
  source 1..13: mol_type = other DNA
ataacttcataca

SEQ ID NO: 90 - DNA, 13 bp, synthetic construct
  source 1..13: mol_type = other DNA
tgtatgaagttat

SEQ ID NO: 91 - DNA, 13 bp, synthetic construct
  source 1..13: mol_type = other DNA
ataactttgtata

SEQ ID NO: 92 - DNA, 13 bp, synthetic construct
  source 1..13: mol_type = other DNA
tatacaaagttat

SEQ ID NO: 93 - DNA, 13 bp, synthetic construct
  source 1..13: mol_type = other DNA
ataacttcgtgca

SEQ ID NO: 94 - DNA, 13 bp, synthetic construct
  source 1..13: mol_type = other DNA
tgcacgaagttat

SEQ ID NO: 95 - DNA, 13 bp, synthetic construct
  source 1..13: mol_type = other DNA
ataactctgtata

SEQ ID NO: 96 - DNA, 13 bp, synthetic construct
  source 1..13: mol_type = other DNA
tatacagagttat

SEQ ID NO: 97 - DNA, 13 bp, synthetic construct
  source 1..13: mol_type = other DNA
ataactctatata

SEQ ID NO: 98 - DNA, 13 bp, synthetic construct
  source 1..13: mol_type = other DNA
tatatagagttat

SEQ ID NO: 99 - DNA, 13 bp, synthetic construct
  source 1..13: mol_type = other DNA
ataactctgtgta

SEQ ID NO: 100 - DNA, 13 bp, synthetic construct
  source 1..13: mol_type = other DNA
tacacagagttat

SEQ ID NO: 101 - DNA, 13 bp, synthetic construct
  source 1..13: mol_type = other DNA
taccgttcgtata

SEQ ID NO: 102 - DNA, 13 bp, synthetic construct
  source 1..13: mol_type = other DNA
tatacgaacggta

SEQ ID NO: 103 - DNA, 34 bp, synthetic construct
  source 1..34: mol_type = other DNA
ataacttcgtataaaaaaaaatatacgaagttat

SEQ ID NO: 104 - DNA, 34 bp, synthetic construct
  source 1..34: mol_type = other DNA
ataacttcgtataaaaaaaattatacgaagttat

SEQ ID NO: 105 - DNA, 34 bp, synthetic construct
  source 1..34: mol_type = other DNA
ataacttcgtataaaaaaatatatacgaagttat

SEQ ID NO: 106 - DNA, 34 bp, synthetic construct
  source 1..34: mol_type = other DNA
ataacttcgtataaaaaaatttatacgaagttat

SEQ ID NO: 107 - DNA, 34 bp, synthetic construct
  source 1..34: mol_type = other DNA
ataacttcgtataaaaaataatatacgaagttat

SEQ ID NO: 108 - DNA, 34 bp, synthetic construct
  source 1..34: mol_type = other DNA
ataacttcgtataaaaaatattatacgaagttat

SEQ ID NO: 109 - DNA, 34 bp, synthetic construct
  source 1..34: mol_type = other DNA
ataacttcgtataaaaaattatatacgaagttat

SEQ ID NO: 110 - DNA, 34 bp, synthetic construct
  source 1..34: mol_type = other DNA
ataacttcgtataaaaaattttatacgaagttat

SEQ ID NO: 111 - DNA, 34 bp, synthetic construct
  source 1..34: mol_type = other DNA
ataacttcgtataaaaataaatatacgaagttat

SEQ ID NO: 112 - DNA, 34 bp, synthetic construct
  source 1..34: mol_type = other DNA
ataacttcgtataaaaataattatacgaagttat

SEQ ID NO: 113 - DNA, 34 bp, synthetic construct
  source 1..34: mol_type = other DNA
ataacttcgtataaaaatatatatacgaagttat

SEQ ID NO: 114 - DNA, 34 bp, synthetic construct
  source 1..34: mol_type = other DNA
ataacttcgtataaaaattaatatacgaagttat

SEQ ID NO: 115 - DNA, 34 bp, synthetic construct
  source 1..34: mol_type = other DNA
ataacttcgtataaaaattattatacgaagttat

SEQ ID NO: 116 - DNA, 34 bp, synthetic construct
  source 1..34: mol_type = other DNA
ataacttcgtataaaaatttatatacgaagttat

SEQ ID NO: 117 - DNA, 34 bp, synthetic construct
  source 1..34: mol_type = other DNA
ataacttcgtataaaaatttttatacgaagttat

SEQ ID NO: 118 - DNA, 34 bp, synthetic construct
  source 1..34: mol_type = other DNA
ataacttcgtataaaataaaatatacgaagttat

SEQ ID NO: 119 - DNA, 34 bp, synthetic construct
  source 1..34: mol_type = other DNA
ataacttcgtataaaataaattatacgaagttat

SEQ ID NO: 120 - DNA, 34 bp, synthetic construct
  source 1..34: mol_type = other DNA
ataacttcgtataaaataatatatacgaagttat

SEQ ID NO: 121 - DNA, 34 bp, synthetic construct
  source 1..34: mol_type = other DNA
ataacttcgtataaaataatttatacgaagttat

SEQ ID NO: 122 - DNA, 34 bp, synthetic construct
  source 1..34: mol_type = other DNA
ataacttcgtataaaatataatatacgaagttat

SEQ ID NO: 123 - DNA, 34 bp, synthetic construct
  source 1..34: mol_type = other DNA
ataacttcgtataaaatatattatacgaagttat

SEQ ID NO: 124 - DNA, 34 bp, synthetic construct
  source 1..34: mol_type = other DNA
ataacttcgtataaaatattatatacgaagttat

SEQ ID NO: 125 - DNA, 34 bp, synthetic construct
  source 1..34: mol_type = other DNA
ataacttcgtataaaatattttatacgaagttat

SEQ ID NO: 126 - DNA, 34 bp, synthetic construct
  source 1..34: mol_type = other DNA
ataacttcgtataaaattaaatatacgaagttat

SEQ ID NO: 127 - DNA, 34 bp, synthetic construct
  source 1..34: mol_type = other DNA
ataacttcgtataaaattaattatacgaagttat

SEQ ID NO: 128 - DNA, 34 bp, synthetic construct; identical to SEQ ID NO: 103
  source 1..34: mol_type = other DNA

SEQ ID NO: 129 - DNA, 34 bp, synthetic construct
  source 1..34: mol_type = other DNA
ataacttcgtataaaattatttatacgaagttat

SEQ ID NO: 130 - DNA, 34 bp, synthetic construct
  source 1..34: mol_type = other DNA
ataacttcgtataaaatttaatatacgaagttat

SEQ ID NO: 131 - DNA, 34 bp, synthetic construct
  source 1..34: mol_type = other DNA
ataacttcgtataaaatttattatacgaagttat

SEQ ID NO: 132 - DNA, 34 bp, synthetic construct
  source 1..34: mol_type = other DNA
ataacttcgtataaaattttatatacgaagttat

SEQ ID NO: 133 - DNA, 34 bp, synthetic construct
  source 1..34: mol_type = other DNA
ataacttcgtataaaattttttatacgaagttat

SEQ ID NO: 134 - DNA, 34 bp, synthetic construct
  source 1..34: mol_type = other DNA
ataacttcgtataaataaaaatatacgaagttat

SEQ ID NO: 135 - DNA, 34 bp, synthetic construct
  source 1..34: mol_type = other DNA
ataacttcgtataaataaaattatacgaagttat

SEQ ID NO: 136 - DNA, 34 bp, synthetic construct
  source 1..34: mol_type = other DNA
ataacttcgtataaataaatatatacgaagttat

SEQ ID NO: 137 - DNA, 34 bp, synthetic construct
  source 1..34: mol_type = other DNA
ataacttcgtataaataaatttatacgaagttat

SEQ ID NO: 138 - DNA, 34 bp, synthetic construct
  source 1..34: mol_type = other DNA
ataacttcgtataaataataatatacgaagttat

SEQ ID NO: 139 - DNA, 34 bp, synthetic construct
  source 1..34: mol_type = other DNA
ataacttcgtataaataatattatacgaagttat

SEQ ID NO: 140 - DNA, 34 bp, synthetic construct
  source 1..34: mol_type = other DNA
ataacttcgtataaataattatatacgaagttat

SEQ ID NO: 141 - DNA, 34 bp, synthetic construct
  source 1..34: mol_type = other DNA
ataacttcgtataaataattttatacgaagttat

SEQ ID NO: 142 - DNA, 34 bp, synthetic construct
  source 1..34: mol_type = other DNA
ataacttcgtataaatataaatatacgaagttat

SEQ ID NO: 143 - DNA, 34 bp, synthetic construct
  source 1..34: mol_type = other DNA
ataacttcgtataaatataattatacgaagttat

SEQ ID NO: 144 - DNA, 34 bp, synthetic construct
  source 1..34: mol_type = other DNA
ataacttcgtataaatatatatatacgaagttat

SEQ ID NO: 145 - DNA, 34 bp, synthetic construct
  source 1..34: mol_type = other DNA
ataacttcgtataaatatatttatacgaagttat

SEQ ID NO: 146 - DNA, 34 bp, synthetic construct
  source 1..34: mol_type = other DNA
ataacttcgtataaatattaatatacgaagttat

SEQ ID NO: 147 - DNA, 34 bp, synthetic construct
  source 1..34: mol_type = other DNA
ataacttcgtataaatattattatacgaagttat

SEQ ID NO: 148 - DNA, 34 bp, synthetic construct
  source 1..34: mol_type = other DNA
ataacttcgtataaatatttatatacgaagttat

SEQ ID NO: 149 - DNA, 34 bp, synthetic construct
  source 1..34: mol_type = other DNA
ataacttcgtataaatatttttatacgaagttat

SEQ ID NO: 150 - DNA, 34 bp, synthetic construct
  source 1..34: mol_type = other DNA
ataacttcgtataaattaaaatatacgaagttat

SEQ ID NO: 151 - DNA, 34 bp, synthetic construct
  source 1..34: mol_type = other DNA
ataacttcgtataaattaaattatacgaagttat

SEQ ID NO: 152 - DNA, 34 bp, synthetic construct
  source 1..34: mol_type = other DNA
ataacttcgtataaattaatatatacgaagttat

SEQ ID NO: 153 - DNA, 34 bp, synthetic construct
  source 1..34: mol_type = other DNA
ataacttcgtataaattaatttatacgaagttat

SEQ ID NO: 154 - DNA, 34 bp, synthetic construct
  source 1..34: mol_type = other DNA
ataacttcgtataaattataatatacgaagttat

SEQ ID NO: 155 - DNA, 34 bp, synthetic construct
  source 1..34: mol_type = other DNA
ataacttcgtataaattatattatacgaagttat

SEQ ID NO: 156 - DNA, 34 bp, synthetic construct
  source 1..34: mol_type = other DNA
ataacttcgtataaattattatatacgaagttat

SEQ ID NO: 157 - DNA, 34 bp, synthetic construct
  source 1..34: mol_type = other DNA
ataacttcgtataaattattttatacgaagttat

SEQ ID NO: 158 - DNA, 34 bp, synthetic construct
  source 1..34: mol_type = other DNA
ataacttcgtataaatttaaatatacgaagttat

SEQ ID NO: 159 - DNA, 34 bp, synthetic construct
  source 1..34: mol_type = other DNA
ataacttcgtataaatttaattatacgaagttat

SEQ ID NO: 160 - DNA, 34 bp, synthetic construct
  source 1..34: mol_type = other DNA
ataacttcgtataaatttatatatacgaagttat

SEQ ID NO: 161 - DNA, 34 bp, synthetic construct
  source 1..34: mol_type = other DNA
ataacttcgtataaatttatttatacgaagttat

SEQ ID NO: 162 - DNA, 34 bp, synthetic construct
  source 1..34: mol_type = other DNA
ataacttcgtataaattttaatatacgaagttat

SEQ ID NO: 163 - DNA, 34 bp, synthetic construct
  source 1..34: mol_type = other DNA
ataacttcgtataaattttattatacgaagttat

SEQ ID NO: 164 - DNA, 34 bp, synthetic construct
  source 1..34: mol_type = other DNA
ataacttcgtataaatttttatatacgaagttat

SEQ ID NO: 165 - DNA, 34 bp, synthetic construct
  source 1..34: mol_type = other DNA
ataacttcgtataaatttttttatacgaagttat

SEQ ID NO: 166 - DNA, 34 bp, synthetic construct
  source 1..34: mol_type = other DNA
ataacttcgtataataaaaaatatacgaagttat

SEQ ID NO: 167 - DNA, 34 bp, synthetic construct
  source 1..34: mol_type = other DNA
ataacttcgtataataaaaattatacgaagttat

SEQ ID NO: 168 - DNA, 34 bp, synthetic construct
  source 1..34: mol_type = other DNA
ataacttcgtataataaaatatatacgaagttat

SEQ ID NO: 169 - DNA, 34 bp, synthetic construct
  source 1..34: mol_type = other DNA
ataacttcgtataataaaatttatacgaagttat

SEQ ID NO: 170 - DNA, 34 bp, synthetic construct
  source 1..34: mol_type = other DNA
ataacttcgtataataaataatatacgaagttat

SEQ ID NO: 171 - DNA, 34 bp, synthetic construct
  source 1..34: mol_type = other DNA
ataacttcgtataataaatattatacgaagttat

SEQ ID NO: 172 - DNA, 34 bp, synthetic construct
  source 1..34: mol_type = other DNA
ataacttcgtataataaattatatacgaagttat

SEQ ID NO: 173 - DNA, 34 bp, synthetic construct
  source 1..34: mol_type = other DNA
ataacttcgtataataaattttatacgaagttat

SEQ ID NO: 174 - DNA, 34 bp, synthetic construct
  source 1..34: mol_type = other DNA
ataacttcgtataataataaatatacgaagttat

SEQ ID NO: 175 - DNA, 34 bp, synthetic construct
  source 1..34: mol_type = other DNA
ataacttcgtataataataattatacgaagttat

SEQ ID NO: 176 - DNA, 34 bp, synthetic construct
  source 1..34: mol_type = other DNA
ataacttcgtataataatatatatacgaagttat

SEQ ID NO: 177 - DNA, 34 bp, synthetic construct
  source 1..34: mol_type = other DNA
ataacttcgtataataatatttatacgaagttat

SEQ ID NO: 178 - DNA, 34 bp, synthetic construct
  source 1..34: mol_type = other DNA
ataacttcgtataataattaatatacgaagttat

SEQ ID NO: 179 - DNA, 34 bp, synthetic construct
  source 1..34: mol_type = other DNA
ataacttcgtataataattattatacgaagttat

SEQ ID NO: 180 - DNA, 34 bp, synthetic construct
  source 1..34: mol_type = other DNA
ataacttcgtataataatttatatacgaagttat

SEQ ID NO: 181 - DNA, 34 bp, synthetic construct
  source 1..34: mol_type = other DNA
ataacttcgtataataatttttatacgaagttat

SEQ ID NO: 182 - DNA, 34 bp, synthetic construct
  source 1..34: mol_type = other DNA
ataacttcgtataatataaaatatacgaagttat

SEQ ID NO: 183 - DNA, 34 bp, synthetic construct
  source 1..34: mol_type = other DNA
ataacttcgtataatataaattatacgaagttat

SEQ ID NO: 184 - DNA, 34 bp, synthetic construct
  source 1..34: mol_type = other DNA
ataacttcgtataatataatatatacgaagttat

SEQ ID NO: 185 - DNA, 34 bp, synthetic construct
  source 1..34: mol_type = other DNA
ataacttcgtataatataatttatacgaagttat

SEQ ID NO: 186 - DNA, 34 bp, synthetic construct
  source 1..34: mol_type = other DNA
ataacttcgtataatatataatatacgaagttat

SEQ ID NO: 187 - DNA, 34 bp, synthetic construct
  source 1..34: mol_type = other DNA
ataacttcgtataatatatattatacgaagttat

SEQ ID NO: 188 - DNA, 34 bp, synthetic construct
  source 1..34: mol_type = other DNA
ataacttcgtataatatattatatacgaagttat

SEQ ID NO: 189 - DNA, 34 bp, synthetic construct
  source 1..34: mol_type = other DNA
ataacttcgtataatatattttatacgaagttat

SEQ ID NO: 190 - DNA, 34 bp, synthetic construct
  source 1..34: mol_type = other DNA
ataacttcgtataatattaaatatacgaagttat

SEQ ID NO: 191 - DNA, 34 bp, synthetic construct
  source 1..34: mol_type = other DNA
ataacttcgtataatattaattatacgaagttat

SEQ ID NO: 192 - DNA, 34 bp, synthetic construct
  source 1..34: mol_type = other DNA
ataacttcgtataatattatatatacgaagttat

SEQ ID NO: 193 - DNA, 34 bp, synthetic construct
  source 1..34: mol_type = other DNA
ataacttcgtataatattatttatacgaagttat

SEQ ID NO: 194 - DNA, 34 bp, synthetic construct
  source 1..34: mol_type = other DNA
ataacttcgtataatatttaatatacgaagttat

SEQ ID NO: 195 - DNA, 34 bp, synthetic construct
  source 1..34: mol_type = other DNA
ataacttcgtataatatttattatacgaagttat

SEQ ID NO: 196 - DNA, 34 bp, synthetic construct
  source 1..34: mol_type = other DNA
ataacttcgtataatattttatatacgaagttat

SEQ ID NO: 197 - DNA, 34 bp, synthetic construct
  source 1..34: mol_type = other DNA
ataacttcgtataatattttttatacgaagttat

SEQ ID NO: 198 - DNA, 34 bp, synthetic construct
  source 1..34: mol_type = other DNA
ataacttcgtataattaaaaatatacgaagttat

SEQ ID NO: 199 - DNA, 34 bp, synthetic construct
  source 1..34: mol_type = other DNA
ataacttcgtataattaaaattatacgaagttat

SEQ ID NO: 200 - DNA, 34 bp, synthetic construct
  source 1..34: mol_type = other DNA
ataacttcgtataattaaatatatacgaagttat

SEQ ID NO: 201 - DNA, 34 bp, synthetic construct
  source 1..34: mol_type = other DNA
ataacttcgtataattaaatttatacgaagttat

SEQ ID NO: 202 - DNA, 34 bp, synthetic construct
  source 1..34: mol_type = other DNA
ataacttcgtataattaataatatacgaagttat

SEQ ID NO: 203 - DNA, 34 bp, synthetic construct
  source 1..34: mol_type = other DNA
ataacttcgtataattaatattatacgaagttat

SEQ ID NO: 204 - DNA, 34 bp, synthetic construct
  source 1..34: mol_type = other DNA
ataacttcgtataattaattatatacgaagttat

SEQ ID NO: 205 - DNA, 34 bp, synthetic construct
  source 1..34: mol_type = other DNA
ataacttcgtataattaattttatacgaagttat

SEQ ID NO: 206 - DNA, 34 bp, synthetic construct
  source 1..34: mol_type = other DNA
ataacttcgtataattataaatatacgaagttat

SEQ ID NO: 207 - DNA, 34 bp, synthetic construct
  source 1..34: mol_type = other DNA
ataacttcgtataattataattatacgaagttat

SEQ ID NO: 208 - DNA, 34 bp, synthetic construct
  source 1..34: mol_type = other DNA
ataacttcgtataattatatatatacgaagttat

SEQ ID NO: 209 - DNA, 34 bp, synthetic construct
  source 1..34: mol_type = other DNA
ataacttcgtataattatatttatacgaagttat

SEQ ID NO: 210 - DNA, 34 bp, synthetic construct
  source 1..34: mol_type = other DNA
ataacttcgtataattattaatatacgaagttat

SEQ ID NO: 211 - DNA, 34 bp, synthetic construct
  source 1..34: mol_type = other DNA
ataacttcgtataattattattatacgaagttat

SEQ ID NO: 212 - DNA, 34 bp, synthetic construct
  source 1..34: mol_type = other DNA
ataacttcgtataattatttatatacgaagttat

SEQ ID NO: 213 - DNA, 34 bp, synthetic construct
  source 1..34: mol_type = other DNA
ataacttcgtataattatttttatacgaagttat

SEQ ID NO: 214 - DNA, 34 bp, synthetic construct
  source 1..34: mol_type = other DNA
ataacttcgtataatttaaaatatacgaagttat

SEQ ID NO: 215 - DNA, 34 bp, synthetic construct
  source 1..34: mol_type = other DNA
ataacttcgtataatttaaattatacgaagttat

SEQ ID NO: 216 - DNA, 34 bp, synthetic construct
  source 1..34: mol_type = other DNA
ataacttcgtataatttaatatatacgaagttat

SEQ ID NO: 217 - DNA, 34 bp, synthetic construct
  source 1..34: mol_type = other DNA
ataacttcgtataatttaatttatacgaagttat

SEQ ID NO: 218 - DNA, 34 bp, synthetic construct
  source 1..34: mol_type = other DNA
ataacttcgtataatttataatatacgaagttat

SEQ ID NO: 219 - DNA, 34 bp, synthetic construct
  source 1..34: mol_type = other DNA
ataacttcgtataatttatattatacgaagttat

SEQ ID NO: 220 - DNA, 34 bp, synthetic construct
  source 1..34: mol_type = other DNA
ataacttcgtataatttattatatacgaagttat

SEQ ID NO: 221 - DNA, 34 bp, synthetic construct
  source 1..34: mol_type = other DNA
ataacttcgtataatttattttatacgaagttat

SEQ ID NO: 222 - DNA, 34 bp, synthetic construct
  source 1..34: mol_type = other DNA
ataacttcgtataattttaaatatacgaagttat

SEQ ID NO: 223 - DNA, 34 bp, synthetic construct
  source 1..34: mol_type = other DNA
ataacttcgtataattttaattatacgaagttat

SEQ ID NO: 224 - DNA, 34 bp, synthetic construct
  source 1..34: mol_type = other DNA
ataacttcgtataattttatatatacgaagttat

SEQ ID NO: 225 - DNA, 34 bp, synthetic construct
  source 1..34: mol_type = other DNA
ataacttcgtataattttatttatacgaagttat

SEQ ID NO: 226 - DNA, 34 bp, synthetic construct
  source 1..34: mol_type = other DNA
ataacttcgtataatttttaatatacgaagttat

SEQ ID NO: 227 - DNA, 34 bp, synthetic construct
  source 1..34: mol_type = other DNA
ataacttcgtataatttttattatacgaagttat

SEQ ID NO: 228 - DNA, 34 bp, synthetic construct
  source 1..34: mol_type = other DNA
ataacttcgtataattttttatatacgaagttat

SEQ ID NO: 229 - DNA, 34 bp, synthetic construct
  source 1..34: mol_type = other DNA
ataacttcgtataattttttttatacgaagttat

SEQ ID NO: 230 - DNA, 34 bp, synthetic construct
  source 1..34: mol_type = other DNA
ataacttcgtatataaaaaaatatacgaagttat

SEQ ID NO: 231 - DNA, 34 bp, synthetic construct
  source 1..34: mol_type = other DNA
ataacttcgtatataaaaaattatacgaagttat

SEQ ID NO: 232 - DNA, 34 bp, synthetic construct
  source 1..34: mol_type = other DNA
ataacttcgtatataaaaatatatacgaagttat

SEQ ID NO: 233 - DNA, 34 bp, synthetic construct
  source 1..34: mol_type = other DNA
ataacttcgtatataaaaatttatacgaagttat

SEQ ID NO: 234 - DNA, 34 bp, synthetic construct
  source 1..34: mol_type = other DNA
ataacttcgtatataaaataatatacgaagttat

SEQ ID NO: 235 - DNA, 34 bp, synthetic construct
  source 1..34: mol_type = other DNA
ataacttcgtatataaaatattatacgaagttat

SEQ ID NO: 236 - DNA, 34 bp, synthetic construct
  source 1..34: mol_type = other DNA
ataacttcgtatataaaattatatacgaagttat

SEQ ID NO: 237 - DNA, 34 bp, synthetic construct
  source 1..34: mol_type = other DNA
ataacttcgtatataaaattttatacgaagttat

SEQ ID NO: 238 - DNA, 34 bp, synthetic construct
  source 1..34: mol_type = other DNA
ataacttcgtatataaataaatatacgaagttat

SEQ ID NO: 239 - DNA, 34 bp, synthetic construct
  source 1..34: mol_type = other DNA
ataacttcgtatataaataattatacgaagttat

SEQ ID NO: 240 - DNA, 34 bp, synthetic construct
  source 1..34: mol_type = other DNA
ataacttcgtatataaatatatatacgaagttat

SEQ ID NO: 241 - DNA, 34 bp, synthetic construct
  source 1..34: mol_type = other DNA
ataacttcgtatataaatatttatacgaagttat

SEQ ID NO: 242 - DNA, 34 bp, synthetic construct
  source 1..34: mol_type = other DNA
ataacttcgtatataaattaatatacgaagttat

SEQ ID NO: 243 - DNA, 34 bp, synthetic construct
  source 1..34: mol_type = other DNA
ataacttcgtatataaattattatacgaagttat

SEQ ID NO: 244 - DNA, 34 bp, synthetic construct
  source 1..34: mol_type = other DNA
ataacttcgtatataaatttatatacgaagttat

SEQ ID NO: 245 - DNA, 34 bp, synthetic construct
  source 1..34: mol_type = other DNA
ataacttcgtatataaatttttatacgaagttat

SEQ ID NO: 246 - DNA, 34 bp, synthetic construct
  source 1..34: mol_type = other DNA
ataacttcgtatataataaaatatacgaagttat

SEQ ID NO: 247 - DNA, 34 bp, synthetic construct
  source 1..34: mol_type = other DNA
ataacttcgtatataataaattatacgaagttat

SEQ ID NO: 248 - DNA, 34 bp, synthetic construct
  source 1..34: mol_type = other DNA
ataacttcgtatataataatatatacgaagttat

SEQ ID NO: 249 - DNA, 34 bp, synthetic construct
  source 1..34: mol_type = other DNA
ataacttcgtatataataatttatacgaagttat

SEQ ID NO: 250 - DNA, 34 bp, synthetic construct
  source 1..34: mol_type = other DNA
ataacttcgtatataatataatatacgaagttat

SEQ ID NO: 251 - DNA, 34 bp, synthetic construct
  source 1..34: mol_type = other DNA
ataacttcgtatataatatattatacgaagttat

SEQ ID NO: 252 - DNA, 34 bp, synthetic construct
  source 1..34: mol_type = other DNA
ataacttcgtatataatattatatacgaagttat

SEQ ID NO: 253 - DNA, 34 bp, synthetic construct
  source 1..34: mol_type = other DNA
ataacttcgtatataatattttatacgaagttat

SEQ ID NO: 254 - DNA, 34 bp, synthetic construct
  source 1..34: mol_type = other DNA
ataacttcgtatataattaaatatacgaagttat

SEQ ID NO: 255 - DNA, 34 bp, synthetic construct
  source 1..34: mol_type = other DNA
ataacttcgtatataattaattatacgaagttat

SEQ ID NO: 256 - DNA, 34 bp, synthetic construct
  source 1..34: mol_type = other DNA
ataacttcgtatataattatatatacgaagttat

SEQ ID NO: 257 - DNA, 34 bp, synthetic construct
  source 1..34: mol_type = other DNA
ataacttcgtatataattatttatacgaagttat

SEQ ID NO: 258 - DNA, 34 bp, synthetic construct
  source 1..34: mol_type = other DNA
ataacttcgtatataatttaatatacgaagttat

SEQ ID NO: 259 - DNA, 34 bp, synthetic construct
  source 1..34: mol_type = other DNA
ataacttcgtatataatttattatacgaagttat

SEQ ID NO: 260 - DNA, 34 bp, synthetic construct
  source 1..34: mol_type = other DNA
ataacttcgtatataattttatatacgaagttat

SEQ ID NO: 261 - DNA, 34 bp, synthetic construct
  source 1..34: mol_type = other DNA
ataacttcgtatataattttttatacgaagttat

SEQ ID NO: 262 - DNA, 34 bp, synthetic construct
  source 1..34: mol_type = other DNA
ataacttcgtatatataaaaatatacgaagttat

SEQ ID NO: 263 - DNA, 34 bp, synthetic construct
  source 1..34: mol_type = other DNA
ataacttcgtatatataaaattatacgaagttat

SEQ ID NO: 264 - DNA, 34 bp, synthetic construct
  source 1..34: mol_type = other DNA
ataacttcgtatatataaatatatacgaagttat

SEQ ID NO: 265 - DNA, 34 bp, synthetic construct
  source 1..34: mol_type = other DNA
ataacttcgtatatataaatttatacgaagttat

SEQ ID NO: 266 - DNA, 34 bp, synthetic construct
  source 1..34: mol_type = other DNA
ataacttcgtatatataataatatacgaagttat

SEQ ID NO: 267 - DNA, 34 bp, synthetic construct
  source 1..34: mol_type = other DNA
ataacttcgtatatataatattatacgaagttat

SEQ ID NO: 268 - DNA, 34 bp, synthetic construct
  source 1..34: mol_type = other DNA
ataacttcgtatatataattatatacgaagttat

SEQ ID NO: 269 - DNA, 34 bp, synthetic construct
  source 1..34: mol_type = other DNA
ataacttcgtatatataattttatacgaagttat

SEQ ID NO: 270 - DNA, 34 bp, synthetic construct
  source 1..34: mol_type = other DNA
ataacttcgtatatatataaatatacgaagttat

SEQ ID NO: 271 - DNA, 34 bp, synthetic construct
  source 1..34: mol_type = other DNA
ataacttcgtatatatataattatacgaagttat

SEQ ID NO: 272 - DNA, 34 bp, synthetic construct
  source 1..34: mol_type = other DNA
ataacttcgtatatatatatatatacgaagttat

SEQ ID NO: 273 - DNA, 34 bp, synthetic construct
  source 1..34: mol_type = other DNA
ataacttcgtatatatatatttatacgaagttat

SEQ ID NO: 274 - DNA, 34 bp, synthetic construct
  source 1..34: mol_type = other DNA
ataacttcgtatatatattaatatacgaagttat

SEQ ID NO: 275 - DNA, 34 bp, synthetic construct
  source 1..34: mol_type = other DNA
ataacttcgtatatatattattatacgaagttat

SEQ ID NO: 276 - DNA, 34 bp, synthetic construct
  source 1..34: mol_type = other DNA
ataacttcgtatatatatttatatacgaagttat

SEQ ID NO: 277 - DNA, 34 bp, synthetic construct
  source 1..34: mol_type = other DNA
ataacttcgtatatatatttttatacgaagttat

SEQ ID NO: 278 - DNA, 34 bp, synthetic construct
  source 1..34: mol_type = other DNA
ataacttcgtatatattaaaatatacgaagttat

SEQ ID NO: 279 - DNA, 34 bp, synthetic construct
  source 1..34: mol_type = other DNA
ataacttcgtatatattaaattatacgaagttat

SEQ ID NO: 280 - DNA, 34 bp, synthetic construct
  source 1..34: mol_type = other DNA
ataacttcgtatatattaatatatacgaagttat

SEQ ID NO: 281 - DNA, 34 bp, synthetic construct
  source 1..34: mol_type = other DNA
ataacttcgtatatattaatttatacgaagttat

SEQ ID NO: 282 - DNA, 34 bp, synthetic construct
  source 1..34: mol_type = other DNA
ataacttcgtatatattataatatacgaagttat

SEQ ID NO: 283 - DNA, 34 bp, synthetic construct
  source 1..34: mol_type = other DNA
ataacttcgtatatattatattatacgaagttat

SEQ ID NO: 284 - DNA, 34 bp, synthetic construct
  source 1..34: mol_type = other DNA
ataacttcgtatatattattatatacgaagttat

SEQ ID NO: 285 - DNA, 34 bp, synthetic construct
  source 1..34: mol_type = other DNA
ataacttcgtatatattattttatacgaagttat

SEQ ID NO: 286 - DNA, 34 bp, synthetic construct
  source 1..34: mol_type = other DNA
ataacttcgtatatatttaaatatacgaagttat

SEQ ID NO: 287 - DNA, 34 bp, synthetic construct
  source 1..34: mol_type = other DNA
ataacttcgtatatatttaattatacgaagttat

SEQ ID NO: 288 - DNA, 34 bp, synthetic construct
  source 1..34: mol_type = other DNA
ataacttcgtatatatttatatatacgaagttat

SEQ ID NO: 289 - DNA, 34 bp, synthetic construct
  source 1..34: mol_type = other DNA
ataacttcgtatatatttatttatacgaagttat

SEQ ID NO: 290 - DNA, 34 bp, synthetic construct
  source 1..34: mol_type = other DNA
ataacttcgtatatattttaatatacgaagttat

SEQ ID NO: 291 - DNA, 34 bp, synthetic construct
  source 1..34: mol_type = other DNA
ataacttcgtatatattttattatacgaagttat

SEQ ID NO: 292 - DNA, 34 bp, synthetic construct
  source 1..34: mol_type = other DNA
ataacttcgtatatatttttatatacgaagttat

SEQ ID NO: 293 - DNA, 34 bp, synthetic construct
  source 1..34: mol_type = other DNA
ataacttcgtatatatttttttatacgaagttat

SEQ ID NO: 294 - DNA, 34 bp, synthetic construct
  source 1..34: mol_type = other DNA
ataacttcgtatattaaaaaatatacgaagttat

SEQ ID NO: 295 - DNA, 34 bp, synthetic construct
  source 1..34: mol_type = other DNA
ataacttcgtatattaaaaattatacgaagttat

SEQ ID NO: 296 - DNA, 34 bp, synthetic construct
  source 1..34: mol_type = other DNA
ataacttcgtatattaaaatatatacgaagttat

SEQ ID NO: 297 - DNA, 34 bp, synthetic construct
  source 1..34: mol_type = other DNA
ataacttcgtatattaaaatttatacgaagttat

SEQ ID NO: 298 - DNA, 34 bp, synthetic construct
  source 1..34: mol_type = other DNA
ataacttcgtatattaaataatatacgaagttat

SEQ ID NO: 299 - DNA, 34 bp, synthetic construct
  source 1..34: mol_type = other DNA
ataacttcgtatattaaatattatacgaagttat

SEQ ID NO: 300 - DNA, 34 bp, synthetic construct
  source 1..34: mol_type = other DNA
ataacttcgtatattaaattatatacgaagttat

SEQ ID NO: 301 - DNA, 34 bp, synthetic construct
  source 1..34: mol_type = other DNA
ataacttcgtatattaaattttatacgaagttat

SEQ ID NO: 302 - DNA, 34 bp, synthetic construct
  source 1..34: mol_type = other DNA
ataacttcgtatattaataaatatacgaagttat

SEQ ID NO: 303 - DNA, 34 bp, synthetic construct
  source 1..34: mol_type = other DNA
ataacttcgtatattaataattatacgaagttat

SEQ ID NO: 304 - DNA, 34 bp, synthetic construct
  source 1..34: mol_type = other DNA
ataacttcgtatattaatatatatacgaagttat

SEQ ID NO: 305 - DNA, 34 bp, synthetic construct
  source 1..34: mol_type = other DNA
ataacttcgtatattaatatttatacgaagttat

SEQ ID NO: 306 - DNA, 34 bp, synthetic construct
  source 1..34: mol_type = other DNA
ataacttcgtatattaattaatatacgaagttat

SEQ ID NO: 307 - DNA, 34 bp, synthetic construct
  source 1..34: mol_type = other DNA
ataacttcgtatattaattattatacgaagttat

SEQ ID NO: 308 - DNA, 34 bp, synthetic construct
  source 1..34: mol_type = other DNA
ataacttcgtatattaatttatatacgaagttat

SEQ ID NO: 309 - DNA, 34 bp, synthetic construct
  source 1..34: mol_type = other DNA
ataacttcgtatattaatttttatacgaagttat

SEQ ID NO: 310 - DNA, 34 bp, synthetic construct
  source 1..34: mol_type = other DNA
ataacttcgtatattataaaatatacgaagttat

SEQ ID NO: 311 - DNA, 34 bp, synthetic construct
  source 1..34: mol_type = other DNA
ataacttcgtatattataaattatacgaagttat

SEQ ID NO: 312 - DNA, 34 bp, synthetic construct
  source 1..34: mol_type = other DNA
ataacttcgtatattataatatatacgaagttat

SEQ ID NO: 313 - DNA, 34 bp, synthetic construct
  source 1..34: mol_type = other DNA
ataacttcgtatattataatttatacgaagttat

SEQ ID NO: 314 - DNA, 34 bp, synthetic construct
  source 1..34: mol_type = other DNA
ataacttcgtatattatataatatacgaagttat

SEQ ID NO: 315 - DNA, 34 bp, synthetic construct
  source 1..34: mol_type = other DNA
ataacttcgtatattatatattatacgaagttat

SEQ ID NO: 316 - DNA, 34 bp, synthetic construct
  source 1..34: mol_type = other DNA
ataacttcgtatattatattatatacgaagttat

SEQ ID NO: 317 - DNA, 34 bp, synthetic construct
  source 1..34: mol_type = other DNA
ataacttcgtatattatattttatacgaagttat

SEQ ID NO: 318 - DNA, 34 bp, synthetic construct
  source 1..34: mol_type = other DNA
ataacttcgtatattattaaatatacgaagttat

SEQ ID NO: 319 - DNA, 34 bp, synthetic construct
  source 1..34: mol_type = other DNA
ataacttcgtatattattaattatacgaagttat

SEQ ID NO: 320 - DNA, 34 bp, synthetic construct
  source 1..34: mol_type = other DNA
ataacttcgtatattattatatatacgaagttat

SEQ ID NO: 321 - DNA, 34 bp, synthetic construct
  source 1..34: mol_type = other DNA
ataacttcgtatattattatttatacgaagttat

SEQ ID NO: 322 - DNA, 34 bp, synthetic construct
  source 1..34: mol_type = other DNA
ataacttcgtatattatttaatatacgaagttat

SEQ ID NO: 323 - DNA, 34 bp, synthetic construct
  source 1..34: mol_type = other DNA
ataacttcgtatattatttattatacgaagttat

SEQ ID NO: 324 - DNA, 34 bp, synthetic construct
  source 1..34: mol_type = other DNA
ataacttcgtatattattttatatacgaagttat

SEQ ID NO: 325 - DNA, 34 bp, synthetic construct
  source 1..34: mol_type = other DNA
ataacttcgtatattattttttatacgaagttat

SEQ ID NO: 326 - DNA, 34 bp, synthetic construct
  source 1..34: mol_type = other DNA
ataacttcgtatatttaaaaatatacgaagttat

SEQ ID NO: 327 - DNA, 34 bp, synthetic construct
  source 1..34: mol_type = other DNA
ataacttcgtatatttaaaattatacgaagttat

SEQ ID NO: 328 - DNA, 34 bp, synthetic construct
  source 1..34: mol_type = other DNA
ataacttcgtatatttaaatatatacgaagttat

SEQ ID NO: 329 - DNA, 34 bp, synthetic construct
  source 1..34: mol_type = other DNA
ataacttcgtatatttaaatttatacgaagttat

SEQ ID NO: 330 - DNA, 34 bp, synthetic construct
  source 1..34: mol_type = other DNA
ataacttcgtatatttaataatatacgaagttat

SEQ ID NO: 331 - DNA, 34 bp, synthetic construct
  source 1..34: mol_type = other DNA
ataacttcgtatatttaatattatacgaagttat

SEQ ID NO: 332 - DNA, 34 bp, synthetic construct
  source 1..34: mol_type = other DNA
ataacttcgtatatttaattatatacgaagttat

SEQ ID NO: 333 - DNA, 34 bp, synthetic construct
  source 1..34: mol_type = other DNA
ataacttcgtatatttaattttatacgaagttat

SEQ ID NO: 334 - DNA, 34 bp, synthetic construct
  source 1..34: mol_type = other DNA
ataacttcgtatatttataaatatacgaagttat

SEQ ID NO: 335 - DNA, 34 bp, synthetic construct
  source 1..34: mol_type = other DNA
ataacttcgtatatttataattatacgaagttat

SEQ ID NO: 336 - DNA, 34 bp, synthetic construct
  source 1..34: mol_type = other DNA
ataacttcgtatatttatatatatacgaagttat

SEQ ID NO: 337 - DNA, 34 bp, synthetic construct
  source 1..34: mol_type = other DNA
ataacttcgtatatttatatttatacgaagttat

SEQ ID NO: 338 - DNA, 34 bp, synthetic construct
  source 1..34: mol_type = other DNA
ataacttcgtatatttattaatatacgaagttat

SEQ ID NO: 339 - DNA, 34 bp, synthetic construct
  source 1..34: mol_type = other DNA
ataacttcgtatatttattattatacgaagttat

SEQ ID NO: 340 - DNA, 34 bp, synthetic construct
  source 1..34: mol_type = other DNA
ataacttcgtatatttatttatatacgaagttat

SEQ ID NO: 341 - DNA, 34 bp, synthetic construct
  source 1..34: mol_type = other DNA
ataacttcgtatatttatttttatacgaagttat

SEQ ID NO: 342 - DNA, 34 bp, synthetic construct
  source 1..34: mol_type = other DNA
ataacttcgtatattttaaaatatacgaagttat

SEQ ID NO: 343 - DNA, 34 bp, synthetic construct
  source 1..34: mol_type = other DNA
ataacttcgtatattttaaattatacgaagttat

SEQ ID NO: 344 - DNA, 34 bp, synthetic construct
  source 1..34: mol_type = other DNA
ataacttcgtatattttaatatatacgaagttat

SEQ ID NO: 345 - DNA, 34 bp, synthetic construct
  source 1..34: mol_type = other DNA
ataacttcgtatattttaatttatacgaagttat

SEQ ID NO: 346 - DNA, 34 bp, synthetic construct
  source 1..34: mol_type = other DNA
ataacttcgtatattttataatatacgaagttat

SEQ ID NO: 347 - DNA, 34 bp, synthetic construct
  source 1..34: mol_type = other DNA
ataacttcgtatattttatattatacgaagttat

SEQ ID NO: 348 - DNA, 34 bp, synthetic construct
  source 1..34: mol_type = other DNA
ataacttcgtatattttattatatacgaagttat

SEQ ID NO: 349 - DNA, 34 bp, synthetic construct
  source 1..34: mol_type = other DNA
ataacttcgtatattttattttatacgaagttat

SEQ ID NO: 350 - DNA, 34 bp, synthetic construct
  source 1..34: mol_type = other DNA
ataacttcgtatatttttaaatatacgaagttat

SEQ ID NO: 351 - DNA, 34 bp, synthetic construct
  source 1..34: mol_type = other DNA
ataacttcgtatatttttaattatacgaagttat

SEQ ID NO: 352 - DNA, 34 bp, synthetic construct
  source 1..34: mol_type = other DNA
ataacttcgtatatttttatatatacgaagttat

SEQ ID NO: 353 - DNA, 34 bp, synthetic construct
  source 1..34: mol_type = other DNA
ataacttcgtatatttttatttatacgaagttat

SEQ ID NO: 354 - DNA, 34 bp, synthetic construct
  source 1..34: mol_type = other DNA
ataacttcgtatattttttaatatacgaagttat

SEQ ID NO: 355 - DNA, 34 bp, synthetic construct
  source 1..34: mol_type = other DNA
ataacttcgtatattttttattatacgaagttat

SEQ ID NO: 356 - DNA, 34 bp, synthetic construct
  source 1..34: mol_type = other DNA
ataacttcgtatatttttttatatacgaagttat

SEQ ID NO: 357 - DNA, 34 bp, synthetic construct
  source 1..34: mol_type = other DNA
ataacttcgtatatttttttttatacgaagttat